(19) United States
(12) Patent Application Publication
(10) Pub. No.: US 2024/0358289 A1
(43) Pub. Date: Oct. 31, 2024

(54) OVERLAPPING DATA STREAMS AND SENSOR TRANSITIONS

(71) Applicant: Dexcom, Inc., San Diego, CA (US)

(72) Inventors: Subhan M. KHAN, San Diego, CA (US); Jonathan M. HUGHES, San Diego, CA (US); Arunachalam PANCH SANTHANAM, San Diego, CA (US); Arturo GARCIA, San Diego, CA (US); Liang WANG, San Diego, CA (US); Yuxi ZHANG, San Diego, CA (US); Jason R. CALABRESE, San Diego, CA (US)

(21) Appl. No.: 18/647,671

(22) Filed: Apr. 26, 2024

Related U.S. Application Data
(60) Provisional application No. 63/499,121, filed on Apr. 28, 2023.

(51) Int. Cl. A61B 5/145; A61B 5/00

(52) U.S. Cl. CPC A61B 5/14546; A61B 5/14532; A61B 5/742

(57) ABSTRACT

Overlapping data streams and sensor transitions is described. In accordance with the described techniques, a first data stream of first analyte measurements is received, at a computing device and during an overlap period, from a first analyte sensor worn by a user and a second data stream of second analyte measurements is received, at the computing device and during the overlap period, from a second analyte sensor worn by the user. The first data stream and the second data stream are received concurrently during the overlap period. A combined analyte measurement is generated based on the first analyte measurements and the second analyte measurements. In one or more implementations, the combined analyte measurement is generated as a weighted average of the first analyte measurements and the second analyte measurements. The combined analyte measurement is then output on a display of the computing device.

### CROSS-REFERENCE TO RELATED APPLICATIONS

This application claims benefit of and priority to U.S. Provisional Application No. 63/499,121, filed Apr. 28, 2023, which is hereby assigned to the assignee hereof and hereby expressly incorporated by reference herein in its entirety as if fully set forth below and for all applicable purposes.

### BACKGROUND

Diabetes is a metabolic condition affecting hundreds of millions of people. For these people, monitoring blood glucose levels and regulating those levels to be within an acceptable range is important not only to mitigate long-term issues such as heart disease and vision loss, but also to avoid the effects of hyperglycemia and hypoglycemia. Maintaining blood glucose levels within an acceptable range can be challenging, as this level is almost constantly changing over time and in response to everyday events, such as eating or exercising. Advances in medical technologies have enabled development of various systems for monitoring blood glucose, including continuous glucose monitoring (CGM) systems, which measure and record glucose concentrations in substantially real-time. CGM systems are important tools for users of these systems to ensure that measured glucose values are within the acceptable range.

Many glucose monitoring systems utilize wearable devices which include sensors that can be inserted into the skin of a user to monitor glucose. Oftentimes these sensors are disposable and designed to operate for a predetermined amount of time (e.g., ten days), after which the sensor must be replaced with a new sensor. When a new sensor is inserted into the user's skin, the sensor may require a significant amount of time (e.g., two hours) to “warmup” before the sensor is able to consistently produce accurate measurements. During this time period, i.e., during the warmup period, glucose measurements produced using a newly inserted glucose sensor may not be accurate and/or may not be consistently accurate. Toward an end of the sensor's designed life, it may also be unable or less able to continue consistently producing accurate measurements.

Due to the unreliability of glucose sensors during the warmup period and at an end of life, many conventional glucose monitoring systems simply refrain from displaying any type of glucose data during those periods. However, the lack of available glucose data during those periods can result in glucose alerts and alarms being disabled, which can lead to health risks for users who rely on glucose data to make important decisions, such as when to administer insulin. Consequently, users may resort to using painful finger sticks during at the end of a sensor's life and during a warmup period of a subsequent sensor in order to monitor their glucose levels. Resorting to finger sticks can also be inconvenient or annoying and may limit the activities those users can undertake.

### SUMMARY

To overcome these problems, overlapping data streams and sensor transitions is described. In accordance with the described techniques, a first data stream of first analyte measurements is received, at a computing device and during an overlap period, from a first analyte sensor worn by a user and a second data stream of second analyte measurements is received, at the computing device and during the overlap period, from a second analyte sensor worn by the user. The first data stream and the second data stream are received concurrently during the overlap period. A combined analyte measurement is generated based on the first analyte measurements and the second analyte measurements. In one or more implementations, the combined analyte measurement is generated as a weighted average of the first analyte measurements and the second analyte measurements. The combined analyte measurement is then output on a display of the computing device.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ overlapping data streams and sensor transitions as described herein.

FIG. 2 depicts an example implementation of an analyte monitoring device in greater detail.

FIG. 3 depicts an example of a system to generate predictions for overlapping data streams and sensor transitions.

FIG. 4 depicts a non-limiting example of a user interface prompting a user to apply an analyte monitoring device for overlapping data streams and sensor transitions.

FIG. 5 depicts a non-limiting example of a user interface prompting a user to remove an analyte monitoring device in connection with overlapping data streams and sensor transitions.

FIG. 6 depicts a non-limiting example of a user interface notifying a user about a predicted event in connection with overlapping data streams and sensor transitions.

FIG. 7 depicts a non-limiting example of a series of user interfaces displaying plots of a user's analyte level over time including during an overlap period of analyte monitoring devices.

FIG. 8 depicts a non-limiting example of a user interface outputting a notification about an impact of behavior on an analyte level in connection with overlapping data streams and sensor transitions.

FIG. 9 depicts a procedure in an example implementation of overlapping streams and sensor transitions.

FIG. 10 depicts a procedure in an additional example implementation of overlapping streams and sensor transitions.

FIG. 11 illustrates an example of a system including various components of an example device that can be implemented as any type of computing device as described and/or utilized with reference to FIGS. 1-10 to implement embodiments of the techniques described herein.

### DETAILED DESCRIPTION

Often, a quality of measurements produced using an analyte sensor degrades as the analyte sensor approaches its “end of life,” and such degradation can begin to occur at varying times for separate analyte sensors due to a variety of factors. As this quality degrades, the measurements may become less consistently accurate in terms of corresponding to an actual level of analyte in the person. This may be observed in a variety of ways, such as when the produced measurements, relative to an actual analyte level, skew lower, skew higher, become more variable, and so forth. These and other issues with analyte measurements may also be present at a beginning of life of an analyte sensor, such as while the sensor is “warming up,” for example, while portions of an analyte sensor that have been inserted into the person suitably hydrate or otherwise prepare to produce analyte measurements. Due to such issues, a person may not be able to unconditionally rely on the analyte measurements produced by such sensors near their end of life and/or near warm up periods (e.g., sensor transitions) to safely and effectively manage a health condition, such as type 1 diabetes. This may result in periods of time around sensor transitions, using conventional techniques, where the person may not be able to use an analyte monitoring device to make treatment decisions for such a health condition. As such, the person may be at a greater risk of experiencing adverse outcomes during sensor transitions with conventional approaches.

Overlapping data streams and sensor transitions are described herein. In accordance with the described techniques, an overlap system is configured to improve analyte measurement quality (e.g., accuracy) and reduce gaps in reliable analyte monitoring with overlapping transitions between sensors, such as when a person applies a second analyte sensor while still wearing a first analyte sensor and then removes the first analyte sensor after some period of time wearing both the first and second analyte sensors concurrently. By way of contrast to conventional approaches, the overlap system monitors transitioning analyte monitoring devices and leverages multivariate data-driven predictions and corrections to output improved analyte measurements and other information that is useable to safely and effectively manage health conditions.

In one or more implementations, the overlap system receives analyte measurements (e.g., glucose measurements) from two different sensors being worn by a user at the same time during an “overlap period”. Broadly, an “overlap period” occurs when a first analyte monitoring device is applied to a person and a second analyte monitoring device is applied to the person such that the first and second analyte monitoring devices are worn by the person at the same time, and may be operable (e.g., producing analyte measurements concurrently). The overlap period ends when at least one of the analyte monitoring devices worn concurrently is removed from the person. The overlap period may vary in length for any amount of time, ranging, for example, from a few seconds to more than a day.

Notably, a quality of measurements produced by an analyte sensor may degrade as the sensor approaches its end of life. In contrast, a newly applied (e.g., inserted) analyte sensor may require an amount of time (e.g., two hours, 30 minutes, etc.) before the measurements produced are consistently accurate measurements. Thus, during a beginning portion of the overlap period the analyte measurements produced by the newly inserted analyte sensor may not be accurate and/or may not be consistently accurate as the newly applied sensor warms up, whereas during an end portion of the overlap period the analyte measurements produced by the first analyte sensor may not be accurate due to degradation of the sensor as it approaches end of life. In general, the overlap system is configured to improve the information output in relation to the overlap period. In this way, the information output may be used to effectively and safely guide treatment decisions for managing one or more health conditions and/or to build an improved (e.g., more accurate) record of the user's physiological state over time.

In one or more implementations, the overlap system monitors a first analyte monitoring device to determine a time to apply a second analyte monitoring device. Based on this determined time, the overlap system prompts the user to apply a new analyte monitoring device. During the overlap period the overlap system monitors both the analyte monitoring device and the analyte monitoring device to determine when to remove the analyte monitoring device. Based on this determined time, the overlap system prompts the user to remove the analyte monitoring device.

In addition to determining when to apply and remove analyte monitoring devices, the overlap system may also generate various other determinations. In one or more implementations, for example, the overlap system determines whether analyte measurements from the first analyte monitoring device or analyte measurements from the second (e.g., newer) analyte monitoring device are “better”, for example, are more accurate or more trusted to represent an actual analyte level of the user. The overlap system then outputs one or more notifications based on the determined more accurate measurements, while suppressing output of the “less accurate” measurements from the other analyte monitoring device.

Rather than simply using the analyte measurements that are determined to be more accurate, in one or more variations, the overlap system monitors the first and second analyte monitoring devices to generate a combined analyte measurement that is a combination of the first analyte measurements received from the first analyte monitoring device and the second analyte measurements received from the second analyte monitoring device. For example, the overlap system may generate the combined analyte measurement by determining weights for the first analyte measurements and for the second analyte measurements and by combining corresponding measurements based on the determined weights (e.g., determining a weighted average). In one or more implementations, the overlap system may determine such weights as a relative amount of trust in the analyte measurements produced by the respective analyte monitoring device at the time the analyte measurements are produced. Alternatively or additionally, the overlap system may determine different weights over time.

To account for sensor quality degradation over time, the overlap system may determine a larger weight for the first analyte measurements received from the first analyte monitoring device (e.g., the older device) near a beginning of the overlap period and a smaller weight for the first analyte measurements near an end of the overlap period. In contrast, to account for sensor warmup, the overlap system may determine a smaller weight for the second analyte measurements received from the second analyte monitoring device (e.g., the more recently applied device) near a beginning of the overlap period and a larger weight for the second analyte measurements near the end of the overlap period. To this extent, the first analyte measurements received from the first analyte monitoring device may contribute more toward an analyte measurement that is a combination of the first analyte measurements and the second analyte measurements at a beginning of the overlap period than at the end of the overlap period. Conversely, the second analyte measurements received from the second analyte monitoring device may contribute less toward an analyte measurement that is a combination of the first analyte measurements and the second analyte measurements at a beginning of the overlap period than at the end of the overlap period. Weights determined for the analyte measurements may be based on a variety of factors other than time in accordance with the described techniques, as discussed in more detail below.

In some aspects, the techniques described herein relate to a method including: receiving, at a computing device and during an overlap period, a first data stream of first analyte measurements from a first analyte sensor worn by a user; receiving, at the computing device and during the overlap period, a second data stream of second analyte measurements from a second analyte sensor worn by the user, the first data stream and the second data stream received concurrently during the overlap period; generating a combined analyte measurement based on the first analyte measurements and the second analyte measurements; and outputting, on a display of the computing device, the combined analyte measurement.

In some aspects, the techniques described herein relate to a method, wherein generating the combined analyte measurement includes: applying a first weight to the first analyte measurements received from the first analyte sensor; applying a second weight to the second analyte measurements received from the second analyte sensor; and generating the combined analyte measurement based on the weighted first analyte measurements and the weighted second analyte measurements.

In some aspects, the techniques described herein relate to a method, wherein the first weight is relatively larger towards a beginning of the overlap period and relatively smaller towards an end of the overlap period.

In some aspects, the techniques described herein relate to a method, wherein the second weight is relatively smaller towards a beginning of the overlap period and relatively larger towards an end of the overlap period.

In some aspects, the techniques described herein relate to a method, wherein generating the combined analyte measurement includes determining a sum of the weighted first analyte measurements and the weighted second analyte measurements.

In some aspects, the techniques described herein relate to a method, wherein the generating the combined analyte measurement includes generating a weighted average of the first analyte measurements and the second analyte measurements.

In some aspects, the techniques described herein relate to a method, wherein the first analyte measurements contribute more towards the combined analyte measurement towards a beginning of an overlap period than at an end of the overlap period.

In some aspects, the techniques described herein relate to a method, wherein the second analyte measurements contribute more towards the combined analyte measurement towards an end of an overlap period than at an end of the overlap period.

In some aspects, the techniques described herein relate to a method, wherein the generating the combined analyte measurement further includes: determining which of the first analyte measurements and the second analyte measurements are more accurate; and outputting the more accurate analyte measurements.

In some aspects, the techniques described herein relate to a method, wherein less accurate analyte measurements are not output.

In some aspects, the techniques described herein relate to a method, wherein less accurate analyte measurements are output concurrently with the more accurate analyte measurements, and wherein the less accurate analyte measurements include an indication that they are less accurate.

In some aspects, the techniques described herein relate to a method, wherein the first analyte sensor and the second analyte sensor include glucose sensors.

In some aspects, the techniques described herein relate to a method, wherein at least one of the first analyte sensor and the second analyte sensor include a glucose sensor.

In some aspects, the techniques described herein relate to a method including: receiving, at a computing device, a first data stream of first analyte measurements from a first analyte sensor worn by a user; monitoring the first analyte sensor to determine a time to apply a second analyte sensor; based on the determined time, outputting a first notification prompting the user to apply a second analyte sensor while continuing to wear the first analyte sensor; receiving, at the computing device and during an overlap period, the first data stream of first analyte measurements from the first analyte sensor worn by the user and a second data stream of second analyte measurements from the second analyte sensor worn by the user; during the overlap period, monitoring the first analyte sensor and the second analyte sensor to determine a removal time to remove the first analyte sensor; and based on the determined removal time, outputting a notification prompting the user to remove the first analyte sensor.

In some aspects, the techniques described herein relate to a method, wherein the first notification indicates the first time to apply the second analyte sensor, and wherein the second notification indicates the second time to remove the first analyte sensor.

In some aspects, the techniques described herein relate to a method, wherein the time to apply the second analyte sensor is based at least in part on an amount of time for which the first analyte sensor is designed or certified to be worn.

In some aspects, the techniques described herein relate to a method, wherein the time to apply the second analyte sensor is based at least in part on a sensor quality measure of the first analyte sensor.

In some aspects, the techniques described herein relate to a method, wherein removal time to remove the first analyte sensor is based at least in part on a first sensor quality measure of the first analyte sensor and a second sensor quality measure of the second analyte sensor.

In some aspects, the techniques described herein relate to a system including: a first analyte monitoring device worn by a user, the first analyte monitoring device configured to produce first analyte measurements; a second analyte monitoring device worn by the user, the second analyte monitoring device configured to produce second analyte measurements; an overlap system implemented at a computing device, the overlap system configured to: receive, during an overlap period, the first analyte measurements from the first analyte monitoring device and the second analyte measurements from the second analyte monitoring device; generate a combined analyte measurement based on the first analyte measurements and the second analyte measurements; and outputting, on a display of the computing device, the combined analyte measurement.

In some aspects, the techniques described herein relate to a system, wherein the overlap system is further configured to: prior to the overlap period, output a notification on the display of the computing device instructing the user to apply the second analyte monitoring device; and during the overlap period, output a second notification on the display of the computing device instructing the user to remove the first analyte monitoring device.

In some aspects, the techniques described herein relate to a system including: a wireless communication interface to receive wirelessly and concurrently, during an overlap period, a first data stream of first analyte measurements from a first analyte sensor worn by a user and a second data stream of second analyte measurements from a second analyte sensor worn by the user; one or more processors; and memory storing instructions that are executable by the one or more processors to perform operations including: generating a combined analyte measurement based on the first analyte measurements and the second analyte measurements; and causing output on a display device of the combined analyte measurement.

In some aspects, the techniques described herein relate to a system, further including the display device to output the combined analyte measurement.

In the following discussion, an exemplary environment is first described that may employ the techniques described herein. Examples of implementation details and procedures are then described which may be performed in the exemplary environment as well as other environments. Performance of the exemplary procedures is not limited to the exemplary environment and the exemplary environment is not limited to performance of the exemplary procedures.

### Example Environment

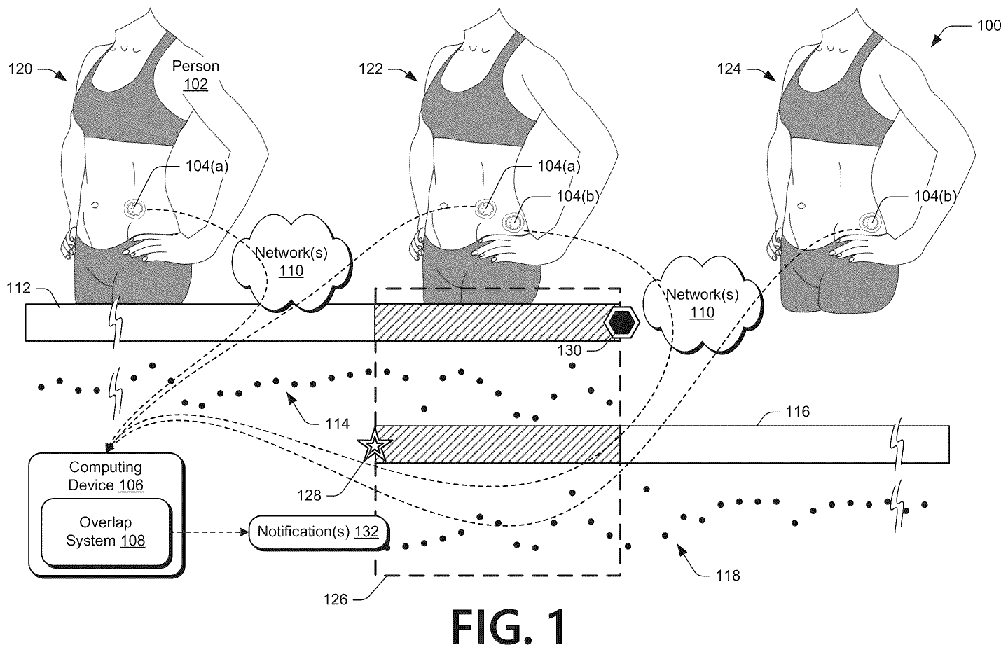

FIG. 1 is an illustration of an environment 100 in an example implementation that is operable to employ overlapping data streams and sensor transitions as described herein. The illustrated environment 100 includes person 102, who is depicted wearing an analyte monitoring device 104, examples of which include analyte monitoring device 104(a) and analyte monitoring device 104(b). The illustrated environment 100 also includes computing device 106, which is depicted having an overlap system 108. The analyte monitoring device 104 and the computing device 106 are communicatively coupled, such as via a network(s) 110. Some, all, or none of the components of the illustrated environment 100 in FIG. 1 may be configured to be disposable (e.g., configured for one-time use by a user) or reusable (e.g., configured for use by a user for multiple analyte monitoring sessions).

The analyte monitoring device 104 and the computing device 106, along with any other optional devices such as medicament delivery systems or other wearables (not shown), may be communicatively coupled in various ways, such as by using one or more wireless communication protocols or techniques. By way of example, the analyte monitoring device 104 and the computing device 106 may communicate with one another using one or more of radio, cellular, WiFi, Bluetooth (e.g., Bluetooth Low Energy links), near-field communication (NFC), and 5G, to name just a few. Thus, the analyte monitoring device 104, the computing device 106, and/or any other optional devices are configured with hardware configured to establish such wireless communication channels and to communicate data over those channels, for example, wireless communication interfaces, network interfaces, one or more antennae, and so forth.

Through such communicative couplings, the analyte monitoring device 104 and the computing devices 106, along with any other optional devices, may form a closed-loop system in one or more implementations. When implemented as a closed-loop system, the combination of devices may be configured to support control of one or more physiological aspects of the person 102 (e.g., glycemic control) in a way that eliminates or reduces user interaction involved with mitigating adverse health events (e.g., glycemic events). Instead, the devices process various information and make various predictions and determinations, such as about when a quality (e.g., accuracy and/or variability) of analyte measurements produced by the analyte monitoring device 104 becomes unsuitable (e.g., at an end of life of the device) for managing one or more health conditions, when the quality of analyte measurements produced by the analyte monitoring device 104 does become suitable (e.g., during warm up of the device) for managing one or more health conditions, activities the user has performed or is performing or will perform based on the analyte measurements, physiological responses (e.g., glycemic responses) to those activities, and therapies needed (if any) to mitigate those physiological responses. In a closed-loop system, the devices may then provide a determined therapy without user interaction, such as by using a medicament delivery system that is controlled to administer medicament to the person 102.

In one or more implementations, the analyte monitoring device 104 is a wearable, such that it is worn by the person 102 while the device performs various operations. Additionally or alternatively, the analyte monitoring device 104 performs one or more operations before or after being worn by the person 102. Broadly, the analyte monitoring device 104 is configured to provide measurements of an analyte of the person 102, such as a glucose level of the person 102. For instance, the analyte monitoring device 104 may be configured with one or more analyte sensors (e.g., one or more glucose sensors) that detect one or more signals indicative of the analyte in the person 102 and enable generation of analyte measurements or estimations (e.g., estimated analyte values). Those analyte measurements (e.g., glucose measurements) may correspond to or otherwise be packaged for communication to one or more of the computing devices 106 or a medicament delivery system as analyte data, examples of which include first data stream 112 which includes first analyte measurements 114 and second data stream 116 which includes second analyte measurements 118.

Although a wearable analyte monitoring device is discussed herein, it is to be appreciated that overlapping data streams and sensor transitions may be used with other configurations of analyte monitoring devices, such as non-wearable analyte devices (e.g., blood glucose meters requiring finger sticks), implants, patches, and so forth. Additionally or alternatively, overlapping data streams and sensor transitions may be used in connection with transitions of sensors that produce data about different physiological phenomena from an analyte, such as multiple analytes, temperature, electrical signals, photonic events, heart rate, heart rate variability, and/or blood pressure.

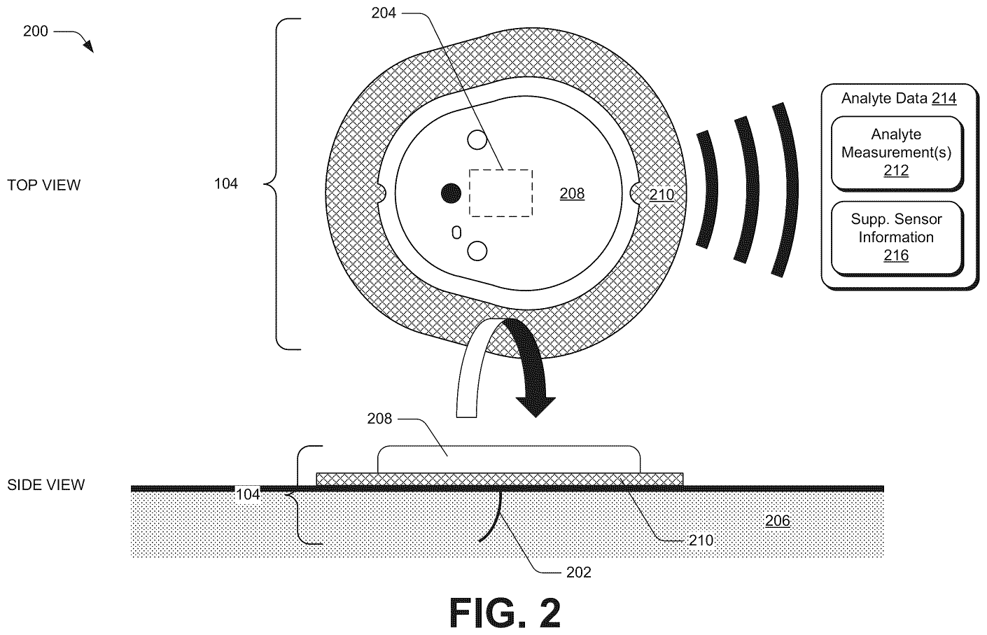

In some embodiments, the analyte monitoring device 104 is a glucose monitoring system. As an example, the analyte monitoring device 104 may be configured as a continuous glucose monitoring (“CGM”) system, e.g., a wearable CGM system. As used herein, the term “continuous” when used in connection with analyte monitoring may refer to an ability of a device to produce analyte measurements substantially continuously, such that the device may be configured to produce the analyte measurements at regular or irregular intervals of time (e.g., approximately every hour, approximately every 30 minutes, approximately every 5 minutes, and so forth), responsive to establishing a communicative coupling with a different device (e.g., when a computing device 106 establishes a wireless connection with the analyte monitoring device 104 to retrieve one or more of the measurements), and so forth. In other implementations, however, the analyte monitoring device 104 may not be “continuous”, and instead provides analyte measurements when requested. For example, the analyte monitoring device 104 may produce a current analyte measurement responsive to a request from the computing device 106. This request may be initiated in a variety of different ways, such as responsive to user input to a user interface (e.g., of an application) displayed by the computing device 106 (and/or a different device), responsive to placement of the computing device 106 within a threshold proximity of the analyte monitoring device 104, responsive to the computing device 106 making physical contact with the analyte monitoring device 104, responsive to a request from an application implemented at the computing device 106, and so forth. This functionality along with further aspects of the configuration of the analyte monitoring device 104 are discussed in more detail in relation to FIG. 2.

In addition to producing analyte measurements 114, 118, the analyte monitoring device 104 may also transmit the produced analyte measurements to the computing device 106 and/or to an analyte monitoring platform or health monitoring platform. The analyte monitoring device 104 may communicate the data in real-time, for example, as it is produced using an analyte sensor. Alternatively or in addition, the analyte monitoring device 104 may communicate the data to a computing device 106 at predefined intervals of time. For example, the analyte monitoring device 104 may be configured to communicate one or more analyte measurements to the computing device 106 approximately every five minutes. It should be appreciated that an interval at which the analyte measurements are communicated by the analyte monitoring device 104 may be different from the examples above without departing from the spirit or scope of the described techniques. In other implementations, one or more analyte measurements are communicated by the analyte monitoring device 104 when requested. For example, the analyte monitoring device 104 device may communicate a current analyte measurement to the computing device 106 responsive to a request from the computing device 106. This request may be initiated in a variety of different ways, such as responsive to user input to a user interface (e.g., of an application) displayed by the computing device 106 (and/or a different device), responsive to placement of the computing device 106 within a threshold proximity of the analyte monitoring device 104, responsive to the computing device 106 making physical contact with the analyte monitoring device 104, responsive to a request from an application implemented at the computing device, and so forth. The data may be communicated by the analyte monitoring device 104 to a computing device 106 according to other bases in accordance with the described techniques.

Additionally, the computing device 106 may maintain one or more of the analyte measurements 114, 118 and/or other data (e.g., about the person 102 and/or a population of users) from various sources at least temporarily, for example, in a storage device (not shown) of the computing device 106. The analyte measurements and such other data may also be maintained in storage of the computing device 106 and/or storage of a different device along with other associated data, such as with corresponding timestamps and/or identifiers of data packets in which communicated, to name just a few.

The computing device 106 may be configured in a variety of ways without departing from the spirit or scope of the described techniques. By way of example and not limitation, the computing device 106 may be configured as a mobile device (e.g., a mobile phone or tablet device), a wearable device (e.g., a smart watch, one or more rings, bracelet or wristband, mouthguard, contact lenses, smart glasses, chest strap, ear buds, clothing, or headphones, etc.), a desktop computer, a laptop computer, and so forth. In one or more implementations, the computing device 106 may be configured as a dedicated device associated with an analyte monitoring platform and/or other health platform, for example, with functionality to obtain analyte measurements from the analyte monitoring device 104, perform various computations in relation to the analyte measurements, display information related to the analyte measurements and the analyte monitoring platform or the health monitoring platform, communicate the analyte measurements to such a platform, and so forth.

Additionally, the computing device 106 may be representative of more than one device in accordance with the described techniques. In one or more scenarios, for instance, the computing device 106 may correspond to both a wearable device (e.g., a smart watch) and a mobile phone. In such scenarios, both of these devices may be capable of performing at least some of the same operations, such as to receive the analyte measurements from the analyte monitoring device 104, receive different sensor data from various other sources, generate sensor data using onboard or associated sensors, communicate data via the network(s) 110 (e.g., to an analyte- or health-monitoring platform), display information related to the data, display or otherwise output information (e.g., notifications) related to overlapping data streams and sensor transitions, facilitate control of a medicament delivery system, and so forth. Alternatively or in addition, different devices may have different capabilities that other devices do not have or that are limited to specified devices through computing instructions.

In accordance with the described techniques, the overlap system 108 is configured to improve analyte measurement quality (e.g., accuracy) and reduce gaps in reliable analyte monitoring with overlapping transitions between sensors, such as when the person 102 applies a second analyte sensor while still wearing a first analyte sensor and then removes the first analyte sensor after some period of time wearing both the first and second analyte sensors concurrently.

Often, a quality of measurements produced using an analyte sensor degrades as the analyte sensor approaches its “end of life,” and such degradation may begin to occur at varying times for separate analyte sensors due to a variety of factors. As this quality degrades, for instance, the measurements may become less consistently accurate in terms of corresponding to an actual level of analyte in the person. This may be observed in a variety of ways, such as when the produced measurements, relative to an actual analyte level, skew lower, skew higher, become more variable, and so forth. These and other issues with analyte measurements may also be present at a beginning of life of an analyte sensor, such as while the sensor is “warming up,” for example, while portions of an analyte sensor that have been inserted into the person suitably hydrate or otherwise prepare to produce analyte measurements. Due to such issues, a person may not be able to unconditionally rely on the analyte measurements produced by such sensors near their end of life and/or near warm up periods (e.g., sensor transitions) to safely and effectively manage a health condition, such as type 1 diabetes. This may result in periods of time around sensor transitions, using conventional techniques, where the person may not be able to use an analyte monitoring device to make treatment decisions for such a health condition. As such, the person may be at a greater risk of experiencing adverse outcomes during sensor transitions with conventional approaches. By way of contrast to conventional approaches, the overlap system 108 monitors transitioning analyte monitoring devices and leverages multivariate data-driven predictions and corrections to output improved analyte measurements and other information that is useable to safely and effectively manage health conditions.

The described techniques are discussed in relation to different stages illustrated in environment 100, such as a first stage 120, a second stage 122, and a third stage 124. By way of example, the chronological order of these stages may correspond to the first stage 120, followed by the second stage 122, and followed by the third stage 124. In the first stage 120, the person 102 is depicted wearing the analyte monitoring device 104(a) which includes a first analyte sensor. In the second stage 122, the person 102 is depicted wearing both the analyte monitoring device 104(a) and the analyte monitoring device 104(b) concurrently. This may correspond to a period of time when the analyte monitoring device 104(b) is applied to the person 102 while still wearing the analyte monitoring device 104(a) and before the analyte monitoring device 104(a) is removed from the person 102. This period of time is an overlap period 126, which may be defined by application 128 of the analyte monitoring device 104(b) (e.g., a new analyte monitoring device) and removal 130 of the analyte monitoring device 104(a) (e.g., an analyte monitoring device already worn when the new analyte monitoring device is applied).

It should be appreciated that an “overlap period” occurs when a first analyte monitoring device is applied to a person and a second analyte monitoring device is applied to the person such that the first and second analyte monitoring devices are worn by the person at the same time, and may be operable (e.g., producing analyte measurements concurrently). The overlap period ends when at least one of the analyte monitoring devices worn concurrently is removed from the person. The overlap period 126 may vary in length for any amount of time, ranging, for example, from a few seconds to more than a day. In the third stage 124, the person 102 is depicted wearing the analyte monitoring device 104(b) which includes a second analyte sensor. As shown, in the first stage 120, the person 102 only wears the analyte monitoring device 104(a) and not the analyte monitoring device 104(b). Similarly, as shown, in the third stage 124, the person 102 only wears the analyte monitoring device 104(b) and not the analyte monitoring device 104(a).

In one or more implementations, the analyte monitoring device 104(a) and the analyte monitoring device 104(b) are completely separate devices. For example, the analyte monitoring device 104(a) and the analyte monitoring device 104(b) may have a same design and be configured in a same manner, but they may be manufactured as separate devices such that they correspond to a first physical device and a separate, second physical device. For instance, the analyte monitoring device 104(a) may include a first analyte sensor and a first transmitter, and the analyte monitoring device 104(b) may include a second analyte sensor and a second transmitter, which are separate from the first analyte sensor and the first transmitter but have a same configuration as the first analyte sensor and transmitter. Alternatively or additionally, the analyte monitoring device 104(a) and the analyte monitoring device 104(b) may be separate devices configured differently, for example, different model numbers, having different types of components from one another. In such scenarios, though, separate devices having different designs and configurations nevertheless may be configured to communicate analyte measurements to the computing device 106.

As noted above, the first data stream 112 includes the first analyte measurements 114. In this example, the analyte monitoring device 104(a) produces the first analyte measurements 114, for example, using one or more analyte sensors of the analyte monitoring device 104(a). In a similar fashion, the second data stream 116 includes the second analyte measurements 118, and the analyte monitoring device 104(b) produces the second analyte measurements 118, for example, using one or more analyte sensors of the analyte monitoring device 104(b). The first analyte measurements 114 are communicated by the analyte monitoring device 104(a) to the computing device 106 as the first data stream 112 using one or more short-range wireless communication technologies. Likewise, the second analyte measurements 118 are communicated by the analyte monitoring device 104(b) to the computing device 106 as the second data stream 116, which also uses one or more short-range wireless communication technologies. Thus, during the overlap period 126 both the analyte monitoring device 104(a) and the analyte monitoring device 104(b) may communicate analyte measurements to the computing device 106, for example, in the first data stream 112 and the second data stream 116, respectively. Thus, the computing device 106 receives the first data stream 112 and the second data stream 116 concurrently during the overlap period 126.

The first analyte measurements 114 and the second analyte measurements 118 are illustrated separately from the first data stream 112 and the second data stream 116 to visually convey how measurements of the person 102's analyte may vary over time—even though the first analyte measurements 114 and the second analyte measurements 118 are included in the first data stream 112 and the second data stream 116, respectively. By illustrating the first analyte measurements 114 and the second analyte measurements 118 separately and as dots, the illustrated environment 100 indicates how measurements of an analyte of the person 102 may vary over time. The illustrated dots may also more closely represent how the computing device 106 outputs (e.g., displays) the person 102's glucose via a user interface, for example, as analyte traces.

Notably, the first analyte measurements 114 and the second analyte measurements 118 that correspond to the overlap period 126 have been illustrated with more variability than the analyte measurements outside (e.g., before and after) the overlap period 126. This represents that in one or more scenarios, a quality of measurements produced by an analyte sensor may degrade as the sensor approaches its end of life and also that a newly applied (e.g., inserted) analyte sensor may require an amount of time (e.g., two hours, 30 minutes, etc.) before the measurements produced are consistently accurate measurements. During at least a portion of the overlap period 126, such as during an initial warmup period of the analyte monitoring device 104(b) immediately following insertion, analyte measurements produced using the analyte monitoring device 104(b) may not be accurate and/or may not be consistently accurate.

In some embodiments, the overlap system 108 is configured to improve the information output in relation to the overlap period 126. In this way, the information output may be used to effectively and safely guide treatment decisions for managing one or more health conditions and/or to build an improved (e.g., more accurate) record of a physiological state of the person 102 over time. To do so, the overlap system 108 may use one or more machine learning models to generate various predictions and/or determinations from a variety of data, including analyte measurements produced using analyte sensors. The overlap system 108 may also output notification(s) 132 based on those predictions and/or determinations, such as by outputting the notification(s) 132 via a user interface of a computer application displayed by a display device of the computing device 106, outputting the notification(s) 132 as audible output of a computer application via one or more speakers associated with the computing device 106, outputting the notification(s) 132 by communicating them (e.g., over the network(s) 110) from the computing device 106 to one or more other computing devices, such as servers of an analyte monitoring platform or other health monitoring platform.

In one or more implementations, the overlap system 108 monitors the analyte monitoring device 104(a) to determine a time to apply a new analyte monitoring device, such as the analyte monitoring device 104(b). Based on this determined time, the overlap system 108 outputs a notification(s) 132 prompting the user to apply a new analyte monitoring device, such as by displaying the notification(s) 132 via a user interface on a display of the computing device 106. During the overlap period 126, the overlap system 108 monitors both the analyte monitoring device 104(a) and the analyte monitoring device 104(b) to determine when to remove the analyte monitoring device 104(a). Based on this determined time, the overlap system 108 outputs a notification(s) 132 prompting the user to remove the analyte monitoring device 104(a), such as by displaying the notification(s) 132 via a user interface on a display of the computing device 106.

In addition to determining when to apply and remove analyte monitoring devices, the overlap system 108 may also generate various other determinations. In one or more implementations, for example, the overlap system 108 determines whether one or more analyte measurements from the analyte monitoring device 104(a) or one or more analyte measurements from the analyte monitoring device 104(b) are “better” (e.g., are more accurate or more trusted to represent an actual analyte level of the person 102). In such implementations, the overlap system 108 may also output one or more notification(s) 132 based on the determined more accurate measurements. For example, the overlap system 108 causes the notification(s) 132 to include the analyte measurements that are determined to be better (e.g., more accurate), such that those analyte measurements are output via a user interface displayed by the computing device 106 on a display device and/or such that those analyte measurements are incorporated into (e.g., stored as part of) a digital record of the analyte measurements of the person 102.

In one or more variations, the analyte measurements that are not the “better” measurements (e.g., are less accurate or less trusted than the other measurements to represent the actual analyte level of the person 102), are not included in the notification(s) 132, such that those measurements are not output via a user interface displayed by the computing device 106 on a display device and/or such that those analyte measurements are not incorporated into (e.g., stored as part of) a digital record of the analyte measurements of the person 102. In at least one variation, the analyte measurements that are not the “better” measurements (e.g., the less accurate or inferior analyte measurements) are included in the notification(s) 132, but the notification(s) 132 include an indication that those measurements are less accurate. Thus, the inferior measurements may be displayed via a user interface and/or incorporated into a record of measurements with an indication that they are determined inferior to the measurements produced by a different analyte monitoring device 104.

Rather than simply using the analyte measurements that are determined to be better, in one or more variations, the overlap system 108 monitors the analyte monitoring device 104(a) and the analyte monitoring device 104(b) to generate analyte measurements that are a combination of the first analyte measurements 114 and the second analyte measurements 118. For example, the overlap system 108 may generate analyte measurements by determining weights for the first analyte measurements 114 and for the second analyte measurements 118 and by combining corresponding measurements based on the determined weights (e.g., determining a weighted average). In one or more implementations, the overlap system 108 may determine such weights as a relative amount of trust in the analyte measurements produced by the respective analyte monitoring device 104 at the time the analyte measurements are produced. Alternatively or additionally, the overlap system 108 may determine different weights over time.

For example, the overlap system 108 may determine a larger weight for the first analyte measurements 114 near a beginning of the overlap period 126 and a smaller weight for the first analyte measurements 114 near an end of the overlap period 126. The overlap system 108 may make such a determination based on data indicating that a quality of measurements from the analyte monitoring device 104(a) is degrading and/or based on data indicating that a quality of measurements from other analyte monitoring devices has been observed to degrade after particular amounts of wear/use and the analyte monitoring device 104(a) may be associated with those other analyte monitoring devices, such as based on a same or similar manufacturing lot, patterns observed in the first analyte measurements 114 and other data (e.g., supplemental sensor data) while the analyte monitoring device 104(a) is in use which are similar to patterns and other data of the other analyte monitoring devices, detection of events during wear which are observed to damage or otherwise degrade the hardware and/or operability of an analyte monitoring device, and so forth.

In one or more scenarios, the overlap system 108 may determine a smaller weight for the second analyte measurements 118 near a beginning of the overlap period 126 and a larger weight for the second analyte measurements 118 near the end of the overlap period 126. The overlap system 108 may make such a determination based on one or more observations that during a warmup period, a newly applied analyte monitoring device may not be capable of producing analyte measurements, may produce analyte measurements which do not accurately correspond to an actual analyte level of a person wearing the analyte monitoring device, may produce analyte measurements which are skewed or demonstrate some other known pattern for an amount of time after application, and so forth

To this extent, the first analyte measurements 114 may contribute more toward an analyte measurement that is a combination of the first analyte measurements 114 and the second analyte measurements 118 at a beginning of the overlap period 126 than at the end of the overlap period 126. Conversely, the second analyte measurements 118 may contribute less toward an analyte measurement that is a combination of the first analyte measurements 114 and the second analyte measurements 118 at a beginning of the overlap period 126 than at the end of the overlap period 126. Weights determined for the analyte measurements may be based on a variety of factors other than time in accordance with the described techniques, as discussed in more detail below.

Additionally or alternatively, the overlap system 108 may identify health events of the person 102 in relation to the overlap period 126 (e.g., before they occur, during occurrence, and/or retrospectively) using a variety of data, such as the first analyte measurements 114 from the analyte monitoring device 104(a) (e.g., before and/or during the overlap period 126), the second analyte measurements 118 from the analyte monitoring device 104 (e.g., during and/or after the overlap period 126), other data associated with the person 102 (e.g., activity data and/or data describing health events including health events input by a user), data associated with other analyte monitoring devices (e.g., used by the person 102 in the past and/or used by other users of a user population), data associated with other users of a user population, and so forth.

Although the overlap system 108 is depicted as part of the computing device 106 in the illustrated example, in one or more variations, the overlap system 108 is partly or entirely included as part of one or more other devices and/or systems, such as servers of a web service, for example, an analyte monitoring platform, a health monitoring platform, and so forth. Additionally or alternatively, the overlap system 108 may be included as part of or otherwise accessible to a computer application of the computing device 106. Such a computer application may correspond to a web service, such as an analyte monitoring platform, a health monitoring platform, and so on.

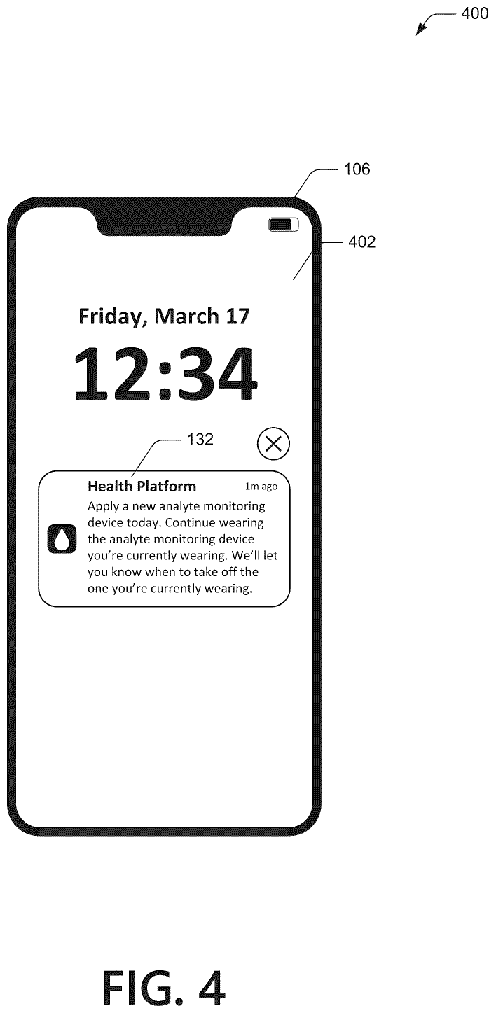

With respect to the notification(s) 132, examples are discussed in more detail below in relation to FIGS. 4-8. Examples of notification(s) 132 include, but are not limited to, a notification to remove the analyte monitoring device 104(a), a notification to apply the analyte monitoring device 104(b), notifications about health events (e.g., glycemic events), notifications about modified and/or replaced measurements, and/or notifications about activities having a health impact (e.g., a glycemic impact). It is to be appreciated that, in some implementations, the overlap system 108 may produce additional and/or different notification(s) 132 without departing from the spirit or scope of the described techniques.

FIG. 2 depicts an example 200 implementation of an analyte monitoring device in greater detail. In particular, the illustrated example 200 includes a top view and a corresponding side view of the analyte monitoring device 104. It is to be appreciated that the analyte monitoring device 104 may vary in implementation from the following discussion in various ways without departing from the spirit or scope of the described techniques.

In this example 200, the analyte monitoring device 104 is illustrated to include an analyte sensor 202 (e.g., a glucose sensor) and a sensor module 204. Here, the analyte sensor 202 is depicted in the side view having been inserted subcutaneously into skin 206 of the person 102. The sensor module 204 is approximated in the top view as a dashed rectangle. The analyte monitoring device 104 also includes a transmitter 208 in the illustrated example 200. Use of the dashed rectangle for the sensor module 204 indicates that it may be housed or otherwise implemented within a housing of the transmitter 208. Antennae and/or other hardware may also be housed or otherwise implemented within the housing of the transmitter 208 and used to enable the transmitter 208 to produce signals for communicating data, including sending and/or receiving data over a wireless connection with the computing device 106 and/or another device such as a medicament delivery device. In example 200, the analyte monitoring device 104 further includes adhesive pad 210.

In operation, the analyte sensor 202 and the adhesive pad 210 may be assembled to form an application assembly, where the application assembly is configured to be applied to the skin 206 so that the analyte sensor 202 is subcutaneously inserted as depicted. In such scenarios, the transmitter 208 may be attached to the assembly after application to the skin 206 via an attachment mechanism (not shown). Alternatively, the transmitter 208 may be incorporated as part of the application assembly, such that the analyte sensor 202, the adhesive pad 210, and the transmitter 208 (with the sensor module 204) may all be applied at once to the skin 206. In one or more implementations, this application assembly is applied to the skin 206 using a separate sensor applicator (not shown). Unlike the finger sticks required by conventional blood glucose meters, user-initiated application of the analyte monitoring device 104 with a sensor applicator is nearly painless and does not require the withdrawal of blood. Moreover, an automatic sensor applicator generally enables the person 102 to embed the analyte sensor 202 subcutaneously into the skin 206 without the assistance of another person, such as a care giver, guardian, clinician, or healthcare provider.

The analyte monitoring device 104 may also be removed by peeling the adhesive pad 210 from the skin 206. It is to be appreciated that the analyte monitoring device 104 and its various components as illustrated are simply one example form factor, and the analyte monitoring device 104 and its components may have different form factors without departing from the spirit or scope of the described techniques.

In operation, the analyte sensor 202 is communicably coupled to the sensor module 204 via at least one communication channel which may be a wireless connection or a wired connection. Communications from the analyte sensor 202 to the sensor module 204 or from the sensor module 204 to the analyte sensor 202 may be implemented actively or passively and these communications may be continuous (e.g., analog) or discrete (e.g., digital).

The analyte sensor 202 may be a device, a molecule, and/or a chemical which changes or causes a change in response to an event which is at least partially independent of the analyte sensor 202. The sensor module 204 is implemented to receive indications of changes to the analyte sensor 202 or caused by the analyte sensor 202. For example, the analyte sensor 202 may include glucose oxidase which reacts with glucose and oxygen to form hydrogen peroxide that is electrochemically detectable by the sensor module 204 which may include an electrode. In this example, the analyte sensor 202 may be configured as or include a glucose sensor configured to detect analytes in blood or interstitial fluid that are indicative of glucose level using one or more measurement techniques. In one or more implementations, the analyte sensor 202 may also be configured to detect analytes in the blood or the interstitial fluid that are indicative of other markers, such as lactate levels, ketones, or ionic potassium, which may improve accuracy in identifying or predicting glucose-based events (e.g., hyperglycemia or hypoglycemia). Additionally or alternatively, the analyte monitoring device 104 may include additional sensors and/or architectures to the analyte sensor 202 to detect those analytes indicative of the other markers.

In another example, the analyte sensor 202 (or an additional sensor of the analyte monitoring device 104—not shown) may include a first and second electrical conductor and the sensor module 204 may electrically detect changes in electric potential across the first and second electrical conductor of the analyte sensor 202. In this example, the sensor module 204 and the analyte sensor 202 are configured as a thermocouple such that the changes in electric potential correspond to temperature changes. In some examples, the sensor module 204 and the analyte sensor 202 are configured to detect a single analyte, such as glucose. In other examples, the sensor module 204 and the analyte sensor 202 are configured to use diverse sensing modes to detect multiple analytes, such as ionic sodium, ionic potassium, carbon dioxide, and glucose. Alternatively or additionally, the analyte monitoring device 104 includes multiple sensors to detect not only one or more analytes (e.g., ionic sodium, ionic potassium, carbon dioxide, glucose, and insulin) but also one or more environmental conditions (e.g., temperature, moisture, and movement). Thus, the sensor module 204 and the analyte sensor 202 (as well as any additional sensors) may detect the presence of one or more analytes, the absence of one or more analytes, and/or changes in one or more environmental conditions. As noted above, the analyte monitoring device 104 may be configured to produce data describing a single analyte (e.g., glucose) or multiple analytes.

In one or more implementations, the sensor module 204 may include one or more processors and one or more memories (not shown). The sensor module 204, by leveraging the one or more processors, may generate analyte measurement(s) 212 based on the communications with the analyte sensor 202 that are indicative of the above-discussed changes. In one or more scenarios, the analyte measurement(s) 212 may correspond to the first analyte measurements 114 and/or the second analyte measurements 118. Based on the above-noted communications from the analyte sensor 202, the sensor module 204 is further configured to generate communicable packages of data that include at least one of the analyte measurement(s) 212. In this example 200, the analyte data 214 represents these packages of data. Additionally or alternatively, the sensor module 204 may configure the analyte data 214 to include additional data, including, by way of example, supplemental sensor information 216. The supplemental sensor information 216 may include a sensor identifier, a sensor status, temperatures that correspond to the analyte measurement(s) 212, measurements of other analytes that correspond to the analyte measurement(s) 212, and so forth. It is to be appreciated that supplemental sensor information 216 may include a variety of data that supplements at least one of the analyte measurement(s) 212 without departing from the spirit or scope of the described techniques.

In implementations where the analyte monitoring device 104 is configured for wireless transmission, the transmitter 208 may transmit the supplemental sensor information 216 as a stream of data or as part of a stream of data to a computing device, such as the first data stream 112 and/or the second data stream 116. Alternatively or additionally, the sensor module 204 may buffer the analyte measurement(s) 212 and/or the supplemental sensor information 216 (e.g., in memory of the sensor module 204 and/or other physical computer-readable storage media of the analyte monitoring device 104) and cause the transmitter 208 to transmit the buffered analyte data 214 later at various regular or irregular intervals, for example, time intervals (approximately every second, approximately every thirty seconds, approximately every minute, approximately every five minutes, approximately every hour, and so on), storage intervals (when the buffered analyte measurement(s) 212 and/or supplemental sensor information 216 reach a threshold amount of data or a number of measurements), and so forth. It should be appreciated that in implementations the analyte monitoring device 104 may vary in numerous ways from the example described above without departing from the spirit or scope of the described techniques.

### Aspects Related to Overlapping Data Streams and Sensor Transitions

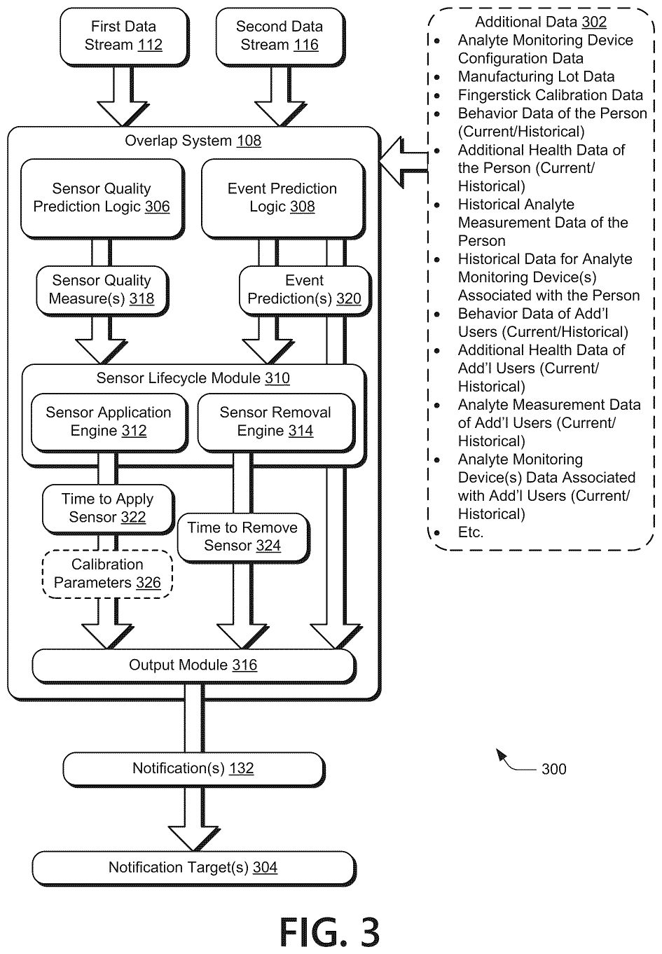

FIG. 3 depicts an example 300 of a system to generate predictions for overlapping data streams and sensor transitions. The illustrated example 300 includes the overlap system 108 depicted and described with respect to FIG. 1.

In example 300, the overlap system 108 is depicted receiving, as input, the first data stream 112, the second data stream 116, and optionally additional data 302. The overlap system 108 is also depicted outputting the notification(s) 132 to notification target(s) 304. In this example, the overlap system 108 includes sensor quality prediction logic 306, event prediction logic 308, sensor lifecycle module 310 which includes sensor application engine 312 and sensor removal engine 314, and output module 316. It is to be appreciated that in variations, the overlap system 108 may include fewer, more, and/or different components without departing from the spirit or scope of the described techniques.

In accordance with the described techniques, the first data stream 112 includes the first analyte measurements 114 and may be received from a first analyte monitoring device, such as the analyte monitoring device 104(a). The second data stream 116 includes the second analyte measurements 118 and may be received from a second analyte monitoring device, such as the analyte monitoring device 104(b). By way of example, the first data stream 112 and/or the second data stream 116 may be received by the overlap system 108 over the network(s) 110, which may include one or more short-range wireless networks and/or long range implemented using any of a variety of wireless technologies, examples of which include Bluetooth, BLE, NFC, 5G, WiFi, and so forth.

In at least one scenario, a portion of the first data stream 112 is produced by the analyte monitoring device 104(a) and communicated to and received by the overlap system 108 before any of the second data stream 116 is produced and communicated to and received by the overlap system 108. Additionally, a subsequent portion of the first data stream 112 is produced by the analyte monitoring device 104(a) and communicated to and received by the overlap system 108 such that it overlaps, as described above, with an initial portion of the second data stream 116. In other words, the initial portion of the second data stream 116 is produced by the analyte monitoring device 104(b) and communicated to and received by the overlap system 108 while the subsequent portion of the first data stream 112 is produced by the analyte monitoring device 104(a) and communicated to and received by the overlap system 108. In one or more scenarios, a subsequent portion of the second data stream 116 is produced by the analyte monitoring device 104(b) and communicated to and received by the overlap system 108 after an entirety of the second data stream 116 is produced and communicated to and received by the overlap system 108. Alternatively, the entirety of the second data stream 116 is produced by the analyte monitoring device 104(b) and communicated to and received by the overlap system 108 while the analyte monitoring device 104(a) is still worn by the person 102, such that no data is produced and communicated by the analyte monitoring device 104(b) after the person 102(a) is removed. This may occur in scenarios where the period of wear of the analyte monitoring device 104(b) is shorter than the period of wear of the analyte monitoring device 104(a), such as when the analyte monitoring device 104(b) is applied to the analyte monitoring device 104 at a time when the analyte monitoring device 104(a) is already worn by the person 102 and is removed before the analyte monitoring device 104(a) is removed.

As noted above, the overlap system 108 also optionally receives the additional data 302. The additional data 302 may be received from a variety of data sources. Examples of such data sources include, but are not limited to, other analyte monitoring devices (e.g., capable of monitoring the same or different analytes), devices that monitoring one or more different physiological signals of the person 102 or other users (e.g., from a population of users), smart watches, smart rings, smart glasses (e.g., augmented reality and/or virtual reality glasses), mobile phones, activity trackers, sleep trackers, meal trackers (e.g., meal journals), health wearables, smart clothing, patches, local storage of the computing device 106, cloud-based storage associated with the computing device 106, an application associated with the overlap system 108 (e.g., installed on the computing device 106), a web service associated with the overlap system 108 (e.g., an analyte monitoring service or other health service), a third-party web service provider, storage associated with the one or more such web services, devices associated with other users of a user population, and/or user data repositories that maintain data about the person 102 and/or other users of a population of users. It is to be appreciated that the additional data may be obtained from these and/or different sources without departing from the spirit or scope of the described techniques.

Further, the additional data 302 may correspond to and/or otherwise describe a variety of aspects associated with overlapping data streams and sensor transitions, and which may be used to improve the information output in relation to the overlap period 126 for safer and more effective management of health conditions. Examples of the additional data 302 include but are not limited to analyte monitoring device configuration data, manufacturing lot data, calibration data (e.g., fingerstick calibration data), behavior data of the person 102 (e.g., including data describing current and historical behavior), health data of the person 102 in addition to analyte measurements (e.g., including data describing current and historical health aspects), historical analyte measurement data of the person 102, historical data describing analyte monitoring devices associated with the person 102 (e.g., analyte monitoring devices previously worn by the person 102), behavior data for other users in a population of users (e.g., including data describing current and historical behavior), health data for other users in a population of users in addition to their analyte measurements (e.g., including data describing current and historical health aspects), analyte measurements or data derived from analyte measurements for other users in a population of users (e.g., including current and historical analyte measurements and/or data derived from the analyte measurements), analyte monitoring device data associated with other users in a population of users (e.g., describing analyte monitoring devices currently and/or previously worn by the other users in the population), and so forth. It is to be appreciated that the additional data 302 may include a variety of data in accordance with the described techniques.

By way of example, device configuration data may include but is not limited to a make and/or model of an analyte monitoring device, device identifier, data describing a configuration of the device (e.g., numbers and what type(s) of sensors), an interval of time for which the device is designed and/or certified to be worn by the person 102, an interval of time that the device is designed to “warm up” after the occurrence of one or more events (e.g., insertion into the person) and during which the device may not provide reliable data and/or measurements, an interval of time (e.g., a window) after some event that the device is designed to provide “optimal” reliability, other aspects and/or limitations for which the device is designed and/or certified, types of wired and/or wireless connections the device is configured to establish and/or maintain, a record of communications produced, firmware version, hardware version, and so on. Device configuration data may describe various aspects of an analyte monitoring device without departing from the spirit or scope of the described techniques.

By way of example, manufacturing data may include but is not limited to a manufacturing lot identifier, manufacturing tolerance data, factory calibration data, averages of one or more physical aspects associated with the analyte monitoring devices in a group (e.g., that are included in a same manufacturing lot), averages of one or more operational aspects associated with the analyte monitoring devices in a group (e.g., average analyte measurement produced when tested using a solution having a known analyte concentration, average temperature measurement produced in an environment having a known temperature, average time interval between measurements produced and/or communicated, average latency between measurement production and communication, average strength of wireless communication signal(s), and so on), standard deviation relative to one or more averages, variability observed in a group based on data used to determine averages and/or standard deviations, metrics derived from a group of analyte monitoring devices having at least one manufacturing commonality (e.g., at least one shared attribute and/or component), and so on. Manufacturing data may describe various aspects associated with a group of analyte monitoring devices sharing at least one manufacturing commonality without departing from the spirit or scope of the described techniques.

By way of example, calibration data (e.g., fingerstick calibration data) may include but is not limited to at least one analyte measurement taken using a blood glucose meter (or other device or technique capable of reliably measuring the analyte at a point in time) and a corresponding analyte measurement produced by an analyte monitoring device (e.g., at substantially a same time), at least one difference between an analyte measurement produced using an analyte monitoring device and a different device capable of measuring the analyte (e.g., a blood glucose meter requiring a fingerstick), adjustments made to calibrate an analyte monitoring device using one or more analyte measurements from a different device capable of measuring the analyte (e.g., a blood glucose meter requiring a fingerstick), an amount of deviation from a factory calibration, one or more metrics describing deviation during a wear period and after being calibrated by a user that an analyte monitoring device produces analyte measurements that are beyond a threshold different from analyte measurements of a different device capable of measuring the analyte (e.g., a blood glucose meter), and so forth. Calibration data may describe various aspects of information used to calibrate an analyte monitoring device and associated with calibrating and/or calibrations of an analyte monitoring device without departing from the spirit or scope of the described techniques.

Behavior data may describe a variety of behaviors of the person and/or one or more additional users of a population of users and/or be otherwise associated those behaviors, and the data may describe current behaviors and/or historical behaviors. By way of example, such behavior data may include but is not limited to activity data (e.g., exercise data), steps data, sleep data, meal data, food data, stress data, sedentary data, productivity data (e.g., tracked as a user's activity with one or more software applications), calendar data, flight data, driving data, hotel data, dating information, social media data, computing device and/or application usage data, gaze data, self-reported data (e.g., digital journaling), breath data, and so forth. Behavior data may describe various aspects of user behavior without departing from the spirit or scope of the described techniques.

Additional health data may describe a variety of aspects of the person and/or one or more additional users of a population that relate to their health and are in addition to analyte measurements, and the data may describe those aspects that relate to their health currently and/or that relate to their health historically. By way of example, such health data may include but is not limited to measurements of different or additional analytes, the occurrence of health events (e.g., heart attack, surgery, illness, injuries, etc.), the presence or absence of health conditions (e.g., as diagnosed by a health care provider and/or self reported), relative health history (e.g., of parents, siblings, grandparents, great grandparents, children, and other blood relatives) which describes health events and/or health conditions of such relatives, measurements of and/or data describing one or more physiological signals (e.g., ECG, EKG), data describing heart activity and/or other cardiovascular aspects (e.g., blood pressure, pulse, etc.), oxygen saturation (SpO2), X-rays, computerized tomography (CT) scans, magnetic resonance imaging (MRI), data produced based on one or more tissue and/or fluid samples, and so forth. Health data may describe various aspects associated with user health without departing from the spirit or scope of the described techniques.

By way of example, analyte monitoring device data may describe various aspects of an analyte monitoring device worn by the person 102 and/or by other users of a population of users, examples of which include but are not limited to sensor and/or manufacturing lot, one or more determined deviations from actual analyte levels (e.g., as measured by a blood glucose meter), a confidence of measurements produced by the analyte monitoring device, an amount of time after some event (e.g., insertion) until measurements produced by the analyte monitoring device satisfy a threshold quality (e.g., until the measurements could reasonably be relied upon to make effective and safe decisions regarding management of a health conditions), an amount of time after some event when the measurements produced by the analyte monitoring device no longer satisfy the threshold quality (e.g., when the measurements could no longer be reasonably relied upon to make effective and safe decisions regarding management of health conditions), a difference between the designed lifespan and the amount of time when the measurements no longer satisfy the threshold quality, an optimal interval of time when an analyte monitoring device produces analyte measurements which satisfy one or more metrics associated with quality (e.g., minimal variability, remain within one or more thresholds of an actual analyte level, are produced at an expected interval, etc.), errors or faults detected during use of an analyte monitoring device, a measure of similarity with one or more other analyte monitoring devices (e.g., determined using one or more clustering techniques such as k-means), and so on. Analyte monitoring device may describe various aspects associated with analyte monitoring devices currently being used, that were previously used, and/or that have not yet been used without departing from the spirit or scope of the described techniques.

In one or more implementations, the sensor quality prediction logic 306 generates sensor quality measure(s) 318, which indicate a quality of analyte measurements produced by an analyte monitoring device. In one or more implementations, for example, the sensor quality prediction logic 306 generates sensor quality measure(s) 318 for an analyte measurement already produced by an analyte monitoring device. For instance, the sensor quality prediction logic 306 receives an analyte measurement from an analyte monitoring device (and one or more preceding measurements from the analyte monitoring device) and generates a sensor quality measure(s) 318 for the analyte measurement. The sensor quality prediction logic 306 may also generate the sensor quality measure(s) 318 for multiple analyte measurements received from the analyte monitoring device. Additionally or alternatively, the sensor quality prediction logic 306 generates a prediction of sensor quality measure(s) 318 for one or more analyte measurements that have not yet been received by the sensor quality prediction logic (e.g., because those measurements have not yet been produced by the analyte measurement device—they will be produced in the future after their quality is predicted).

In accordance with the described techniques, the sensor quality measure(s) 318 indicate a relative quality of analyte measurements, such that the analyte measurements may be compared and/or ranked relative to one another in terms of quality. In at least one variation, a more favorable sensor quality measure (e.g., higher) indicates that the respective analyte measurements more closely and/or more consistently correspond to actual analyte levels and may be more reasonably relied upon to make safe and effective treatment decisions in relation to a health condition. By way of contrast, a less favorable sensor quality measure (e.g., lower) indicates that the respective analyte measurements less closely and/or less consistently correspond to actual analyte levels and are less reasonably relied upon to make safe and effective treatment decisions in relation to a health condition.

The sensor quality measure(s) 318 may be formatted in a variety of ways without departing from the spirit or scope of the described technique, an example of which is normalized values between 0 and 1, where 1 indicates that the analyte measurement or group of analyte measurements correspond to actual analyte levels and may be relied upon for treatment decisions whereas 0 indicates that the analyte measurement or group of analyte measurements do not correspond to actual analyte levels and are not reliability for treatment decisions.

Alternatively or additionally, the sensor quality measure(s) 318 may include one or more measures. In one or more implementations, for example, the sensor quality measure(s) 318 may correspond to or otherwise include noise estimates, such as estimates of noise in one or more analyte measurements already produced by an analyte monitoring device and processed by the sensor quality prediction logic and/or estimates of noise that is predicted to be in one or more subsequent analyte measurements from the analyte monitoring device that have not yet been processed by the sensor quality prediction logic (e.g., analyte measurements that will be produced by the analyte monitoring device in the future). In one or more implementations, the sensor quality measure(s) 318 may correspond to or otherwise indicate progressive sensor decline (PSD) likelihood. For example, the sensor quality measure(s) 318 may indicate PSD likelihood of an analyte monitoring device over time. The sensor quality measure(s) 318 may include or incorporate any number of various measures and/or metrics which are useable to convey a quality of measurements produced by an analyte monitoring device and/or to indicate a relative “trust” in the measurements produced by an analyte monitoring device.

In this way, a quality of measurements produced by one or more analyte monitoring devices and/or a “trust” in measurements produced by such devices may be compared over time. For example, the sensor quality measure(s) 318 for an individual analyte monitoring device may be compared over time, such as at a beginning of a period of wear and at one or more subsequent times during the period of wear. The sensor quality measure(s) 318 may also be compared for analyte measurements produced by different analyte monitoring devices, such as analyte measurements produced by the different devices at corresponding times (e.g., a same date and time).

Consider an example in which, at time tᵢ (e.g., 120 hours into a 240-hour certified life), where i represents a given number of time intervals from application of the analyte monitoring device 104(a) on the person 102, the sensor quality prediction logic 306 generates sensor quality measures(s) 318 for the first analyte measurements 114 produced by the analyte monitoring device 104(a) up to the time tᵢ. In accordance with the described techniques, at time tᵢ, the sensor quality prediction logic 306 also generates predictions of the sensor quality measure(s) 318 for the first analyte measurements 114 after time tᵢ, such as for the first analyte measurements 114 generated at subsequent intervals i of time, such as measurements at times tᵢ₊₁, tᵢ₊₂, . . . , tᵢ₊ₙ. In this way, the overlap system 108 produces information capable of indicating when analyte measurements produced by the analyte monitoring device 104(a) are likely to no longer satisfy a threshold quality (e.g., a suitability for making effective and safe treatment decisions). As discussed below, this information is usable by the overlap system 108 to determine when to apply a new analyte monitoring device, so that the new analyte monitoring device is operable and producing analyte measurements of suitable quality by the time the first analyte measurements 114 are predicted to no longer satisfy a threshold quality. Accordingly, the sensor quality prediction logic 306 may use the first data stream 112 received up to the time tᵢ and optionally at least a portion of the additional data 302 to generate the sensor quality measure(s) 318 for the first analyte measurements 114 received up to the time tᵢ and for the first analyte measurements 114 predicted to be received after the time tᵢ.

The sensor quality prediction logic 306 may generate the sensor quality measure(s) 318 for the analyte monitoring device 104(b) in a similar manner. When the analyte monitoring device 104(b) is applied on the person 102 at time t₀, which may be prior to producing any of the second analyte measurements 118, the sensor quality prediction logic 306 may generate predictions of the sensor quality measure(s) 318 for all of the second analyte measurements 118 to be produced by the analyte monitoring device 104(b) at times subsequent to time t₀, such as times t₁, t₂, . . . , tₙ. In this way, the overlap system 108 produces information capable of indicating when analyte measurements produced by the analyte monitoring device 104(b) are likely to begin satisfying the threshold quality (e.g., a suitability for making effective and safe treatment decisions). As discussed below, this information, along with the information about an “older” or longer-worn analyte monitoring device applied previously, such as the analyte monitoring device 104(a)), is usable by the overlap system 108 to determine when to remove the analyte monitoring device 104(a), such that the analyte measurements produced by the analyte monitoring device 104(a) are of suitable quality to inform health condition management and/or a patient record until the analyte monitoring device 104(a) is removed, but they may not be of suitable quality after the determined removal time. Once analyte measurements are received from the analyte monitoring device 104(b), the sensor quality prediction logic 306 may use the second analyte measurements 118 received up to a time t and optionally at least a portion of the additional data 302 to generate the sensor quality measure(s) 318 for the second analyte measurements 118 received up to the time t and for the second analyte measurements 118 predicted to be received after the time tᵢ.

In operation, the sensor quality prediction logic 306 may be configured as executable code capable of receiving the first data stream 112 (or a portion thereof), the second data stream 116 (or a portion thereof), and/or at least a portion of the additional data 302 as input. In some embodiments, such input may be configured as one or more feature vectors suitable for receipt by a machine-learning model. The sensor quality prediction logic 306 may further be capable of processing such input according to an encoded algorithm, formula, set of rules, and/or machine-learning models, and determining and outputting the sensor quality measure(s) 318. The sensor quality prediction logic 306 may be implemented based on a variety of algorithms, formulas, rules, and/or models without departing from the spirit or scope of the described techniques.

In some embodiments, a machine learning model of the sensor quality prediction logic 306 (or an ensemble of them) may be configured to receive, as input, data from one or more of the first data stream 112 (e.g., one or more of the first analyte measurements 114), data from the second data stream 116 (e.g., one or more of the second analyte measurements 118), and/or at least a portion of the additional data 302. The machine learning model may then determine the sensor quality measure(s) 318 by applying weights to the inputs (e.g., feature vectors representing the above-noted data). The weights may correspond to an underlying model determined using historical data of a user population and/or the person 102. In such implementations, the machine learning model may be configured to output data (e.g., feature vectors) indicative of the sensor quality measure(s) 318. The outputted data (e.g., the feature vectors) may be processed to obtain the sensor quality measure(s) 318.

In one or more implementations, the event prediction logic 308 illustrated in FIG. 3 may generate event prediction(s) 320, which indicate at least of one of an event that is predicted to occur, including when in some examples, or effects of predicted or detected behaviors on analyte measurements. Examples of predicted events include, but are not limited to, a prediction that the person 102 has eaten or will eat (e.g., without receiving explicit input indicating that the user has eaten), a prediction that the person 102 has exercised or will exercise, a prediction that the person 102 has slept or will sleep, a prediction of a potentially dangerous health condition (e.g., a glycemic excursion such as hyperglycemia or hypoglycemia), a prediction of a positive impact on analyte measurements due to one or more behaviors, a prediction of a negative impact on analyte measurements due to one or more behaviors, a prediction of neutral impact on analyte measurements due to one or more behaviors, a likelihood of a dip and recovery (DnR) event. To generate the event prediction(s) 320, the event prediction logic 308 may use one or more of the first data stream 112 (or a portion thereof), the second data stream 116 (or a portion thereof), and/or the additional data 302.

In operation, the event prediction logic 308 may be configured as executable code capable of receiving the first data stream 112 (or a portion thereof), the second data stream 116 (or a portion thereof), and/or at least a portion of the additional data 302 as input. In at least one variation, such input is configured as one or more feature vectors suitable for receipt by a machine-learning model. The event prediction logic 308 may further be capable of processing such input according to an encoded algorithm, formula, set of rules, and/or machine-learning models, and determining and outputting the event prediction(s) 320. The event prediction logic 308 may be implemented based on a variety of algorithms, formulas, rules, and/or models without departing from the spirit or scope of the described techniques.

In at least one variation, a machine learning model of the event prediction logic 308 (or an ensemble of them) is configured to receive as input data from one or more of the first data stream 112 (e.g., one or more of the first analyte measurements 114), data from the second data stream 116 (e.g., one or more of the second analyte measurements 118), and/or at least a portion of the additional data 302. This machine learning model may then determine the event prediction(s) 320 by applying weights to the inputs (e.g., feature vectors representing the above-noted data). Those weights may correspond to an underlying model determined using historical data of a user population and/or the person 102. In such implementations, the machine learning model is configured to output data (e.g., feature vectors) indicative of the event prediction(s) 320. The output data (e.g., the feature vectors) may be processed to obtain the event prediction(s) 320.

As noted above, the sensor quality prediction logic 306 and the event prediction logic 308 may be implemented based on a variety of algorithms, formulas, rules, and/or models. In implementations, where the sensor quality prediction logic 306 and/or the event prediction logic 308 is implemented based on, or otherwise includes, one or more machine learning models, at least one of those models may be “general” to a population. For example, such a model may be trained using training data that describes users of a user population and is trained in order to generate predictions of sensor quality measure(s) 318 for analyte monitoring devices of various users of the user population and/or predictions of event prediction(s) 320 for those various users. Broadly speaking, a model that is “general” to a population may not be trained for a specific end user when it is used in operation to generate predictions for the specific end user, for example, through further training using transfer learning and/or training data of the specific end user. Alternatively or additionally, at least one of the models used by the sensor quality prediction logic 306 and/or the event prediction logic 308 may be specific to an end user. Such a model may be trained using historical data describing and/or associated with the specific end user for which it is trained to generate predictions of sensor quality measure(s) 318 or event prediction(s) 320. In one or more implementations, such user-specific models initially may be “general” to a population and then further trained using transfer learning and/or training data describing the specific user.

In the illustrated example 300, the overlap system 108 is further depicted including the sensor lifecycle module 310, which is depicted with the sensor application engine 312 and the sensor removal engine 314. The sensor lifecycle module 310 is depicted receiving the sensor quality measure(s) 318 and the event prediction(s) 320 as input. To this end, the sensor application engine 312 determines when to apply an analyte monitoring device (i.e., having at least one analyte sensor) based on at least one of the sensor quality measure(s) 318 or the event prediction(s) 320. In one or more implementations, the sensor application engine 312 outputs a time to apply sensor 322 based on the determination of when to apply an analyte monitoring device. By way of contrast, the sensor removal engine 314 determines when to remove an analyte monitoring device (i.e., having at least one analyte sensor) based on at least one of the sensor quality measure(s) 318 or the event prediction(s) 320. In one or more implementations, the sensor removal engine 314 outputs a time to remove sensor 324. In this example 300, the sensor application engine 312 is depicted optionally outputting calibration parameters 326, which may be used to calibrate the analyte monitoring device being next applied to the person 102, e.g., proximate the time to apply sensor 322.

In the context of FIG. 1, for example, the sensor application engine 312 may determine, during the first stage 120, when to apply the analyte monitoring device 104(b) based on the sensor quality measure(s) 318 and/or the event prediction(s) 320. Alternatively or additionally, the sensor application engine 312 determines when to apply the analyte monitoring device 104(b) based on an amount of time for which the analyte monitoring device 104(a) is designed and/or certified to be worn, noise estimates of the analyte measurements produced by the analyte monitoring device 104(a), progressive sensor decline (PSD) likelihood of the analyte monitoring device 104(a), and/or likelihood of DnR event. Based on this determination, the sensor application engine 312 generates the time to apply sensor 322, which indicates the determined time.

In scenarios where the parameters used to calibrate the analyte monitoring device 104(a) are suitable parameters and are determined likely to cause suitable operation of the analyte monitoring device 104(b), the sensor application engine 312 may “pass on” those parameters so that they may be used to calibrate the analyte monitoring device 104(b). The sensor application engine 312 thus optionally outputs the calibration parameters 326 of a previous analyte monitoring device when determined suitable for calibrating a subsequent analyte monitoring device for operation. By way of example, the calibration parameters 326 of the first analyte monitoring device may be communicated (e.g., wirelessly by the overlap system 108 and/or by an application of the computing device 106) to the second analyte monitoring device and loaded into storage of the second analyte monitoring device, so that one or more of the calibration parameters 326 of the first analyte monitoring device are used to calibrate the second analyte monitoring device for operation.

With respect to the time to remove sensor 324, in the context of FIG. 1, the sensor removal engine 314 may determine, during the overlap period 126, when to remove the analyte monitoring device 104(a) based on the sensor quality measure(s) 318 and/or the event prediction(s) 320. Based on this determination, the sensor removal engine 314 generates the time to remove sensor 324, which indicates the determined time.

The output module 316 may be configured to determine the notification(s) 132 to output and to which notification target(s) 304 based on the determinations and/or predictions made by the components of the overlap system 108. In this example, the output module 316 is depicted as receiving the event prediction(s) 320, the time to apply sensor 322, the time to remove sensor 324, and the calibration parameters 326. Alternatively or additionally, the output module 316 receives one or more of the first data stream 112, second data stream 116, and/or at least a portion of the additional data 302, and uses this information to determine the notification(s) 132 to output to which notification target(s) 304. In variations, the output module 316 uses different and/or additional information to determine the notification(s) 132 to output to the notification target(s) 304.

As discussed, example notification(s) 132 may include, but are not limited to, a prompt to apply an analyte monitoring device, a prompt to remove an analyte monitoring device, an alert or indication of a health event (e.g., a glycemic event), an alert or indication of a DnR event, an analyte measurement modification (e.g., correction) or replacement, a notification about an analyte measurement modification or replacement, calibration parameters, an instruction to bypass an analyte monitoring device (e.g., use the measurements of a more “trusted” analyte monitoring device or an analyte monitoring device for which the sensor quality measure(s) 318 are more favorable), a notification about one or more behaviors having an impact on the analyte measured by the analyte monitoring devices (e.g., a glycemic impact), and so on. The notification(s) 132 may include and/or indicate a variety of information without departing from the spirit or scope of the described techniques.

Like the sources of the additional data 302, the notification target(s) 304 vary in accordance with the described techniques. Alternatively or additionally, the notification target(s) 304 may depend, at least in part, on the notification(s) 132 to be output. In one example, for instance, when the notification(s) 132 include calibration parameters 326 (e.g., from the analyte monitoring device 104(a)), the analyte monitoring device 104(b) may correspond to the notification target(s) 304. In another example, when the notification(s) 132 include the time to apply sensor 322, a user interface of an application of the computing device 106 may correspond to the notification target(s) 304. Examples of notification target(s) 304 include but are not limited to user interfaces (e.g., of applications), one or more applications, storage of the computing device 106, an analyte monitoring device, another device (e.g., a medicament delivery device), a web service provider (e.g., an analyte monitoring platform and/or other health platform), a repository of data for a population of users, a computing device or user account associated with a different user, and so forth. The notification target(s) 304 may include and/or correspond to any one or more of a variety of different entities without departing from the spirit or scope of the described techniques.

As noted above, the overlap system 108 may correct and/or modify measurements produced by one or more of the analyte monitoring device 104(a) and/or the analyte monitoring device 104(b). For example, the overlap system 108 may combine a measurement from analyte monitoring device 104(a) with a measurement from analyte monitoring device 104(b) to generate a combined measurement, an example of which is a weighted average as discussed below.

In one or more examples, the overlap system 108 may use analyte measurements from one or more of the first data stream 112 or the second data stream 116 to generate combined analyte measurements for output via a user interface and/or to be maintained in a record of analyte measurements of the person 102. Additionally, the overlap system 108 may generate such combined analyte values to replace one or more analyte measurements of the first data stream 112 and/or the second data stream 116. For example, the overlap system 108 may generate combined analyte values to replace analyte measurements of the first data stream 112 and/or second data stream 116 during the overlap period 126.

In some embodiments, the overlap system 108 determines the combined analyte measurements using one or more weighting techniques, such as weighted averages. For example, the overlap system 108 may associate relatively smaller weights with the second analyte measurements 118 toward a beginning of the overlap period 126 and may associate relatively larger weights with the second analyte measurements 118 closer to an end of the overlap period 126. In one or more implementations, the overlap system 108 may associate progressively larger weights with the second analyte measurements 118 over time. As an example, at a beginning of the overlap period 126, overlap system 108 may assign a weight of 0.9 to the first analyte measurements 114 and a weight of 0.1 to the second analyte measurements 118, and then compute the estimated analyte values as the sum of the weighted first and second analyte measurements. In contrast, towards the end of the overlap period 126, overlap system 108 may assign a weight of 0.1 to the first analyte measurements 114 and a weight of 0.9 to the second analyte measurements 118, and then compute the estimated analyte values as the sum of the weighted first and second analyte measurements.

In operation, newly applied analyte sensors (e.g., glucose sensors) of analyte monitoring devices may be less accurate earlier in a wear period (e.g., during a first day). At least one advantage of the described techniques is that the overlap system 108 is able to smooth a transition from the first analyte measurements 114 produced by the analyte monitoring device 104(a) to the second analyte measurements 118 produced by the analyte monitoring device 104(b). During the second stage 122, for instance, when both the analyte monitoring device 104(a) and the analyte monitoring device 104(b) are applied to the person 102, in one or more variations, the overlap system 108 maintains communication with both of those devices and may share the measurements produced by the analyte monitoring device 104(a) with the analyte monitoring device 104(b) and/or vice versa. In one or more implementations, an algorithm running on the analyte monitoring device 104(b), for example, via a processor/microcontroller and/or by using some other hardware, firmware, and/or software structure(s) capable of executing the algorithm, determines if the measurements produced by the analyte monitoring device 104(a) are useable to calibrate the analyte monitoring device 104(b). Responsive to the overlap system 108 determining that the analyte monitoring device 104(b) produces analyte measurements with less noise than the analyte measurements produced by the analyte monitoring device 104(a), the overlap system 108 outputs notification(s) 132, such as a notification to remove the analyte monitoring device 104(a). Alternatively or additionally, the analyte monitoring device 104(a) outputs one or more of the notification(s) 132 to one or more display devices, such as a phone and/or a smart watch, indicating that the analyte monitoring device 104(b) has become the primary analyte monitoring device (e.g., instead of the analyte monitoring device 104(a)).

FIGS. 4-8, discussed below, provide example of causing output of the notification(s) 132 via user interface. For example, FIG. 4 depicts a non-limiting example 400 of a user interface prompting a user to apply an analyte monitoring device for overlapping data streams and sensor transitions.

As shown, example 400 includes the computing device 106 which is depicted as displaying a user interface 402 via a display device of the computing device 106. In example 400, the user interface 402 presents a notification(s) 132 which includes a prompt to apply a new analyte monitoring device. In the context of FIG. 1, for example, the output module 316 may cause output of a notification(s) 132 prompting a user to apply the analyte monitoring device 104(b) during the first stage 120 while the person 102 is only wearing the analyte monitoring device 104(a). Further, in some embodiments, the output module 316 may cause output of this notification(s) 132 based on receipt of the time to apply sensor 322. In some embodiments, the notification(s) 132 may be output in additional and/or different ways, such as audibly via one or more speakers associated with the computing device 106.

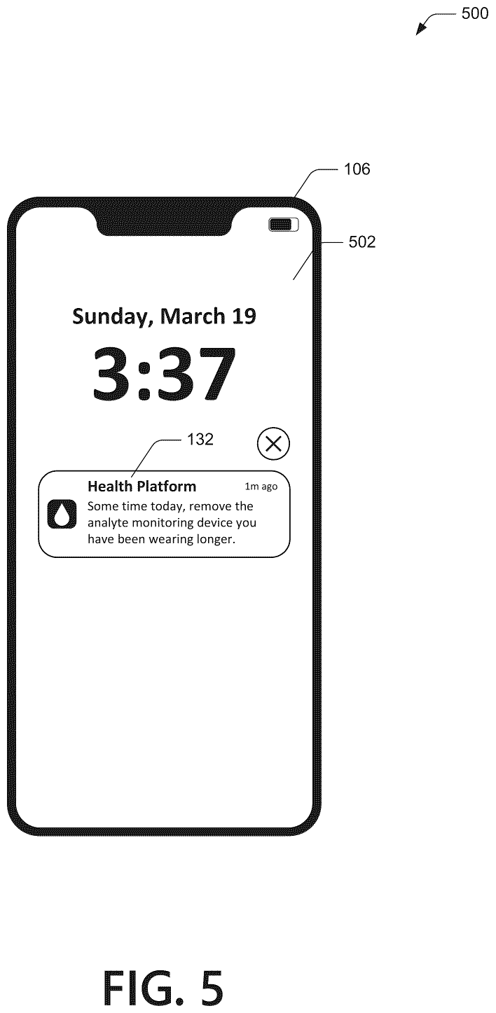

FIG. 5 depicts a non-limiting example 500 of a user interface prompting a user to remove an analyte monitoring device in connection with overlapping data streams and sensor transitions.

As shown, example 500 includes the computing device 106 which is depicted as displaying a user interface 502 via a display device of the computing device 106. In example 500, the user interface 502 presents a notification(s) 132 which includes a prompt to remove a worn analyte monitoring device. In the context of FIG. 1, for example, the output module 316 may cause output of a notification(s) 132 prompting a user to remove the analyte monitoring device 104(a) during the overlap period 126 while the person 102 is wearing both the analyte monitoring device 104(a) and the analyte monitoring device 104(b). In one or more implementations, the notification(s) 132 includes an identifier or other information unique to the device which the overlap system 108 is prompting to remove. Further, in some embodiments, the output module 316 may cause output of the notification(s) 132 based on receipt of the time to remove sensor 324. In some embodiments, the notification(s) 132 may be output in additional and/or different ways, such as audibly via one or more speakers associated with the computing device 106.

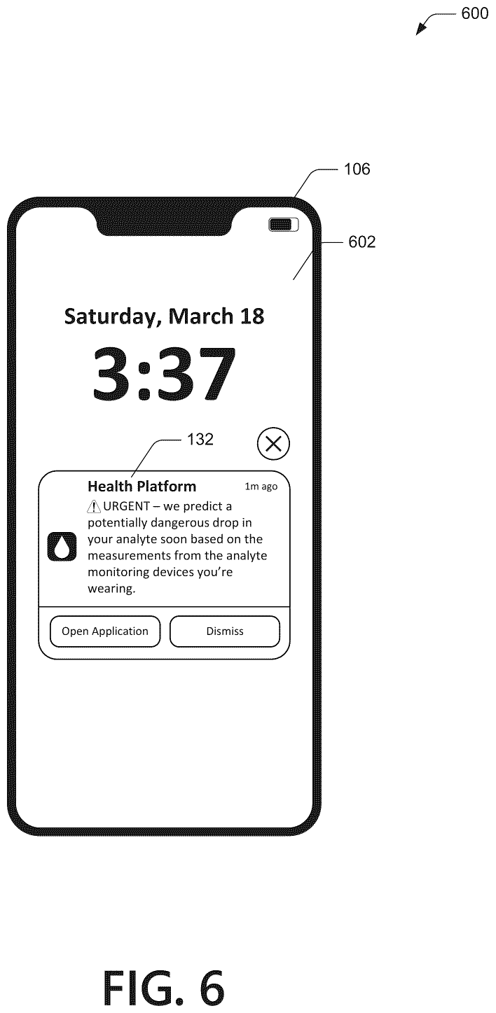

FIG. 6 depicts a non-limiting example 600 of a user interface notifying a user about a predicted event in connection with overlapping data streams and sensor transitions.

As illustrated, example 600 includes the computing device 106 which is depicted as displaying a user interface 602 via a display device of the computing device 106. In example 600, the user interface 602 presents a notification(s) 132 which includes a prediction of an event—in this case, a prediction of glycemic event. In at least one scenario, the output module 316 causes output of this notification(s) 132 based on receipt of an event prediction(s) 320. Further, the event prediction(s) 320 may be predicted, at least in part, based on the second analyte measurements 118 from the analyte monitoring device 104(b), which may be calibrated using the calibration parameters 326 from the analyte monitoring device 104(a). Alternatively or additionally, the event prediction(s) 320 may be predicted using one or more of the first analyte measurements 114 and one or more of the second analyte measurements 118 produced while both the analyte monitoring device 104(a) and the analyte monitoring device 104(b) are being worn during the overlap period 126. In some embodiments, the notification(s) 132 may be output in additional and/or different ways, such as audibly via one or more speakers associated with the computing device 106.

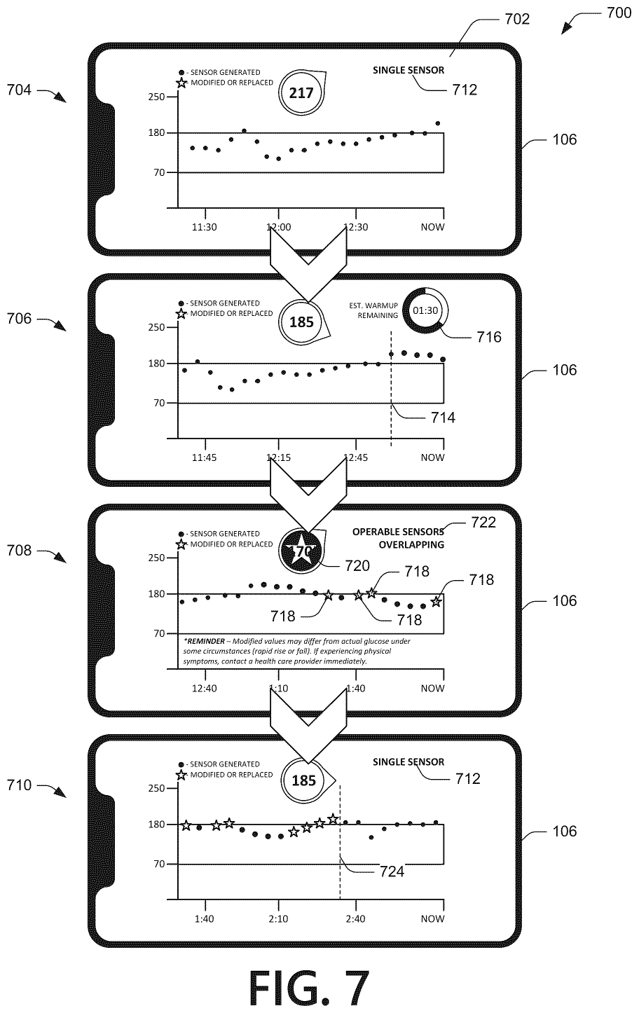

FIG. 7 depicts a non-limiting example 700 of a series of user interfaces displaying plots of a user's analyte level over time including during an overlap period of analyte monitoring devices.

The illustrated example 700 depicts another example of the computing device 106 having a display device. In this example 700, the display device is depicted outputting the user interface 702 at four chronologically sequenced stages, including a first stage 704, a second stage 706, a third stage 708, and a fourth stage 710. The display device of the computing device 106 may correspond to a notification target(s) 304 and the output module 316 may cause output of one or more notification(s) 132 via the user interface 702 displayed by the display device. In example 700, for example, the notification(s) 132 may correspond to one or more modified (e.g., corrected) and or replaced analyte measurements, which may be determined based in part on the sensor quality measure(s) 318 and account for a manufacturing lot of an analyte monitoring device 104.

Each of the stages 704, 706, 708, and 710 plot an analyte over time, for example, based on the first analyte measurements 114 and/or the second analyte measurements 118. The stages 704, 706, 708, and 710 may correspond to different points in time relative to a sensor transition, including an overlap period 126. By way of example, the user interface 702 as displayed at the first stage 704 may be generated and displayed at a time during the first stage 120, for example, when the analyte monitoring device 104(a) is worn by the person 102 and the analyte monitoring device 104(b) is not. In contrast, the user interface 702 as displayed at the second stage 706 and the third stage 708 may be generated and displayed at subsequent times during the overlap period 126, for example, when both the analyte monitoring device 104(a) and the analyte monitoring device 104(b) are worn by the person 102. Further, the user interface 702 as displayed at the fourth stage 710 may be generated and displayed at a time during the third stage 124, for example, when the analyte monitoring device 104(b) is the worn by the person 102 and the analyte monitoring device 104(a) is not.

Accordingly, at the first stage 704, the user interface 702 includes a graph that plots the first analyte measurements 114 over time. Notably, the graph presented via the user interface 702 at the first stage 704 only displays analyte measurements and does not display any modified and/or replaced values. The user interface 702 also includes an indication 712 (e.g., “SINGLE SENSOR”) that the person 102 is wearing only one analyte monitoring device and/or that only one analyte monitoring device is communicably coupled with the computing device 106.

The user interface 702 presented at the second stage 706 corresponds to a time after the analyte monitoring device 104(b) is applied to the person 102 (e.g., inserted into the person 102) and before the device is able to produce reliable analyte measurements. An example of this time is during a warmup period of the analyte monitoring device 104(b). A time that the analyte monitoring device 104(b) is inserted is indicated by graphical element 714 (e.g., a dashed line), which may be displayed as part of the user interface 702 in at least one variation and may not be displayed as part of the user interface 702 in at least one other variation. In example 700, the user interface 702 also includes an indication 716 of a remaining warmup time of the analyte monitoring device 104(b). Since the second analyte measurements 118 may not be reliable during this time, the first analyte measurements 114 from the analyte monitoring device 104(a) may be used (and displayed) and are not modified and/or replaced.

The user interface 702 presented at the third stage 708 corresponds to a time when both the analyte monitoring device 104(a) and the analyte monitoring device 104(b) are worn and after the analyte monitoring device 104(b) is suitably warmed up to provide reliable analyte measurements. At the third stage 708, the user interface 702 includes markers 718 displayed on the graph, which indicate modified and/or replace analyte measurements. Additionally, the user interface 702 includes a graphical emphasis 720 of a current analyte value, indicating that the current analyte value is modified and/or replaced. Further, the user interface 702 includes an indication 722 (e.g., “OPERABLE SENSORS OVERLAPPING”) that the person 102 is wearing at least two analyte monitoring devices and/or that at least two analyte monitoring devices are communicably coupled with the computing device 106.

The user interface 702 presented at the fourth stage 710 corresponds to a time when the analyte monitoring device 104(b) is worn and the analyte monitoring device 104(b) has been removed. A time that the analyte monitoring device 104(a) is removed is indicated by graphical element 724 (e.g., a dashed line), which may be displayed as part of the user interface 702 in at least one variation and may not be displayed as part of the user interface 702 in at least one other variation. Notably, the analyte measurements presented on the graph after the graphical element 724 in terms of time (e.g., to the right), only include produced analyte measurements and do not include any modified and/or replaced values. The user interface 702 again includes the indication 712 (e.g., “SINGLE SENSOR”) that the person 102 is wearing only one analyte monitoring device and/or that only one analyte monitoring device is communicably coupled with the computing device 106.

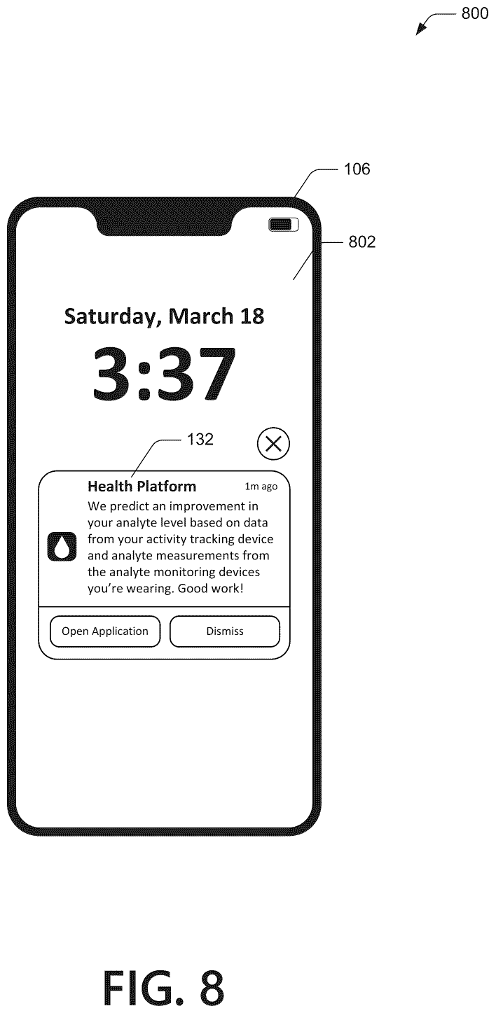

FIG. 8 depicts a non-limiting example 800 of a user interface outputting a notification about an impact of behavior on an analyte level in connection with overlapping data streams and sensor transitions.

The illustrated example 800 includes the computing device 106 which is depicted displaying a user interface 802 via a display device of the computing device 106. In example 800, the user interface 802 presents a notification(s) 132 which includes a prediction of an impact on an analyte level due to one or more behaviors—in this case, a prediction of improved glucose levels based on activity, such as exercise. In at least one scenario, the output module 316 causes output of this notification(s) 132 based on receipt of an event prediction(s) 320. Such an event prediction(s) 320 may be predicted, at least in part, based on the second analyte measurements 118 from the analyte monitoring device 104(b), which may be calibrated using the calibration parameters 326 from the analyte monitoring device 104(a). Alternatively or additionally, the event prediction(s) 320 may be predicted using one or more of the first analyte measurements 114 and one or more of the second analyte measurements 118 produced while both the analyte monitoring device 104(a) and the analyte monitoring device 104(b) are being worn, during the overlap period 126. In variations, the notification(s) 132 may be output in additional and/or different ways, such as audibly via one or more speakers associated with the computing device 106.

### Example Procedures

This section describes examples of procedures for overlapping data streams and sensor transitions. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations the procedures are performed by an overlap system, such as the overlap system 108 that makes use of at least one of the sensor quality prediction logic 306, the event prediction logic 308, the sensor application engine 312, or the sensor removal engine 314.

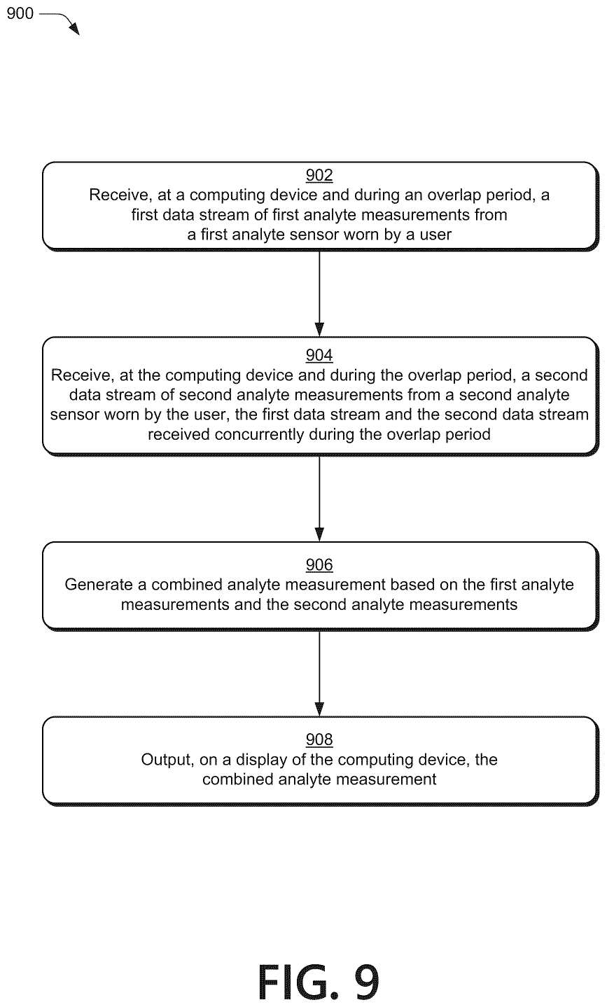

FIG. 9 depicts a procedure 900 in an example implementation of overlapping streams and sensor transitions.

As shown at 902, a first data stream of first analyte measurements is received, at a computing device and during an overlap period, from a first analyte sensor worn by a user (e.g., person 102. Further, as shown at 904, a second data stream of second analyte measurements is received, at the computing device and during the overlap period, from a second analyte sensor worn by the user. In accordance with the principles discussed herein, the first data stream and the second data stream may be received concurrently during the overlap period. By way of example, the computing device 106 receives first data stream 112 which includes the first analyte measurements 114 from the analyte monitoring device 104(a) during an overlap period 126. During the same overlap period 126 the computing device 106 concurrently receives the second data stream 116 which includes the second analyte measurements 118 from the analyte monitoring device 104(b).

As shown at 906, a combined analyte measurement is generated based on the first analyte measurements and the second analyte measurements. By way of example, the overlap system 108 generates a combined analyte measurement that is a combination of the first analyte measurements 114 and the second analyte measurements 118. For example, the overlap system 108 may generate combined analyte measurements by determining weights for the first analyte measurements 114 and for the second analyte measurements 118 and by combining corresponding measurements based on the determined weights (e.g., determining a weighted average). In one or more implementations, the overlap system 108 may determine such weights as a relative amount of trust in the analyte measurements produced by the respective analyte monitoring device 104 at the time the analyte measurements are produced. Alternatively or additionally, the overlap system 108 may determine different weights over time.

For example, the overlap system 108 may determine a larger weight for the first analyte measurements 114 near a beginning of the overlap period 126 and a smaller weight for the first analyte measurements 114 near an end of the overlap period 126. The overlap system 108 may make such a determination based on data indicating that a quality of measurements from the analyte monitoring device 104(a) is degrading and/or based on data indicating that a quality of measurements from other analyte monitoring devices has been observed to degrade after particular amounts of wear/use and the analyte monitoring device 104(a) may be associated with those other analyte monitoring devices, such as based on a same or similar manufacturing lot, patterns observed in the first analyte measurements 114 and other data (e.g., supplemental sensor data) while the analyte monitoring device 104(a) is in use which are similar to patterns and other data of the other analyte monitoring devices, detection of events during wear which are observed to damage or otherwise degrade the hardware and/or operability of an analyte monitoring device, and so forth.

In one or more scenarios, the overlap system 108 may determine a smaller weight for the second analyte measurements 118 near a beginning of the overlap period 126 and a larger weight for the second analyte measurements 118 near the end of the overlap period 126. The overlap system 108 may make such a determination based on one or more observations that during a warmup period, a newly applied analyte monitoring device may not be capable of producing analyte measurements, may produce analyte measurements which do not accurately correspond to an actual analyte level of a person wearing the analyte monitoring device, may produce analyte measurements which are skewed or demonstrate some other known pattern for an amount of time after application, and so forth

To this extent, the first analyte measurements 114 may contribute more toward an analyte measurement that is a combination of the first analyte measurements 114 and the second analyte measurements 118 at a beginning of the overlap period 126 than at the end of the overlap period 126. Conversely, the second analyte measurements 118 may contribute less toward an analyte measurement that is a combination of the first analyte measurements 114 and the second analyte measurements 118 at a beginning of the overlap period 126 than at the end of the overlap period 126. Weights determined for the analyte measurements may be based on a variety of factors other than time in accordance with the described techniques, as discussed in more detail below.

As shown at 908, the combined analyte measurement is output on a display of the computing device. By way of example, the combined analyte measurement is output on a display of computing device 106.

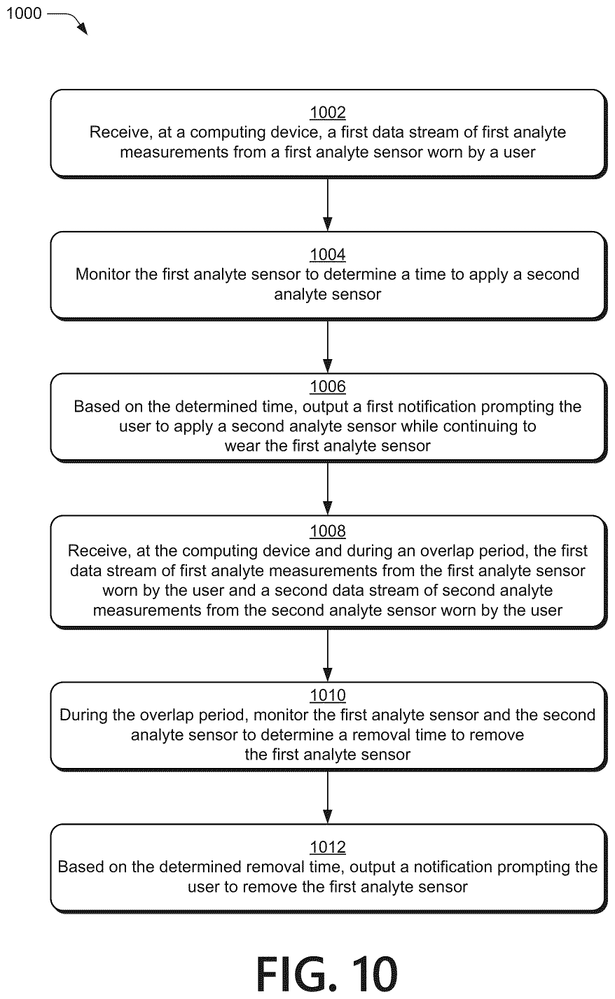

FIG. 10 depicts a procedure 1000 in an additional example implementation of overlapping streams and sensor transitions.

As shown at 1002, a first data stream of first analyte measurements is received, at a computing device, from a first analyte sensor worn by a user. By way of example, computing device 106 receives a first data stream 112 which includes the first analyte measurements 114 from the analyte monitoring device 104(a).

As shown at 1004, the first analyte sensor is monitored to determine a time to apply a second analyte sensor. By way of example, the overlap system 108 monitors the analyte monitoring device 104(a) to determine a time to apply a new analyte monitoring device, such as the analyte monitoring device 104(b).

As shown at 1006, based on the determined time, a first notification prompting the user to apply a second analyte sensor while continuing to wear the first analyte sensor is output. By way of example, based on this determined time, the overlap system 108 outputs a notification(s) 132 prompting the user to apply a new analyte monitoring device, such as by displaying the notification(s) 132 via a user interface on a display of the computing device 106.

As shown at 1008, during an overlap period, the first data stream of first analyte measurements is received at the computing device from the first analyte sensor worn by the user and a second data stream of second analyte measurements is also received at the computing device from the second analyte sensor worn by the user. By way of example, computing device 106 receives first data stream 112 which includes the first analyte measurements 114 from the analyte monitoring device 104(a) during an overlap period 126. During the same overlap period 126 the computing device 106 concurrently receives the second data stream 116 which includes the second analyte measurements 118 from the analyte monitoring device 104(b).

As shown at 1010, during the overlap period, the first analyte sensor and the second analyte sensor are monitored to determine a removal time to remove the first analyte sensor. By way of example, during the overlap period 126, the overlap system 108 monitors both the analyte monitoring device 104(a) and the analyte monitoring device 104(b) to determine when to remove the analyte monitoring device 104(a).

As shown at 1012, based on the determined removal time, a notification is output which prompts the user to remove the first analyte sensor. By way of example, based on this determined time, the overlap system 108 outputs a notification(s) 132 prompting the user to remove the analyte monitoring device 104(a), such as by displaying the notification(s) 132 via a user interface on a display of the computing device 106.

### Example System and Device

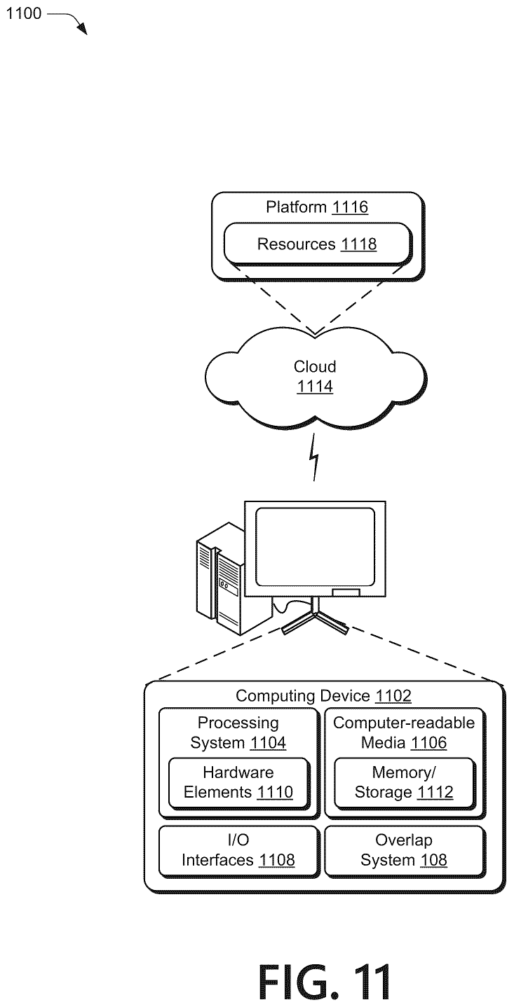

FIG. 11 illustrates an example of a system 1100 that includes an example computing device 1102 that is representative of one or more computing systems and/or devices that may implement the various techniques described above. The computing device 1102 may be, for example, a server of a service provider, a device associated with a client (e.g., a client device), an on-chip system, and/or any other suitable computing device or computing system.

As shown, the computing device 1102 includes a processing system 1104, one or more computer-readable media 1106, one or more I/O interfaces 1108, and the overlap system 108, which may each be communicatively coupled with each other and configured to implement the various techniques described above. Although not shown, the computing device 1102 may further include a system bus or other data and command transfer system that couples the various components, one to another. A system bus may include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1104 is representative of functionality to perform one or more operations using hardware. Accordingly, the processing system 1104 is illustrated as including hardware elements 1110, which may include one or more processors, functional blocks, and so forth. This may include implementation in hardware as an application specific integrated circuit or other logic device formed using one or more semiconductors. The hardware elements 1110 are not limited by the materials from which they are formed or the processing mechanisms employed therein. For example, the one or more processors may be comprised of semiconductor(s) and/or transistors (e.g., electronic integrated circuits (ICs)). In such a context, processor-executable instructions may be electronically-executable instructions.

The computer-readable media 1106 is illustrated as including memory/storage 1112. The memory/storage 1112 represents memory/storage capacity associated with one or more computer-readable media. The memory/storage 1112 may include volatile media (such as random access memory (RAM)) and/or nonvolatile media (such as read only memory (ROM), Flash memory, optical disks, magnetic disks, and so forth). The memory/storage 1112 may include fixed media (e.g., RAM, ROM, a fixed hard drive, and so on) as well as removable media (e.g., Flash memory, a removable hard drive, an optical disc, and so forth). The computer-readable media 1106 may be configured in a variety of other ways as further described below.

Input/output interface(s) 1108 are representative of functionality to allow a user to enter commands and information to computing device 1102, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1102 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 1102. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1102, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1110 and computer-readable media 1106 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1110. The computing device 1102 may be configured to implement particular instructions and/or functions corresponding to the software and/or hardware modules. Accordingly, implementation of a module that is executable by the computing device 1102 as software may be achieved at least partially in hardware, e.g., through use of computer-readable storage media and/or hardware elements 1110 of the processing system 1104. The instructions and/or functions may be executable/operable by one or more articles of manufacture (for example, one or more computing devices 1102 and/or processing systems 1104) to implement techniques, modules, and examples described herein.

The techniques described herein may be supported by various configurations of the computing device 1102 and are not limited to the specific examples of the techniques described herein. This functionality may also be implemented all or in part through use of a distributed system, such as over a “cloud” 1114 via a platform 1116 as described below.

The cloud 1114 includes and/or is representative of a platform 1116 for resources 1118. The platform 1116 abstracts underlying functionality of hardware (e.g., servers) and software resources of the cloud 1114. The resources 1118 may include applications and/or data that may be utilized while computer processing is executed on servers that are remote from the computing device 1102. Resources 1118 may also include services provided over the Internet and/or through a subscriber network, such as a cellular or Wi-Fi network.

The platform 1116 may abstract resources and functions to connect the computing device 1102 with other computing devices. The platform 1116 may also serve to abstract scaling of resources to provide a corresponding level of scale to encountered demand for the resources 1118 that are implemented via the platform 1116. Accordingly, in an interconnected device embodiment, implementation of functionality described herein may be distributed throughout the system 1100. For example, the functionality may be implemented in part on the computing device 1102 as well as via the platform 1116 that abstracts the functionality of the cloud 1114.

### Additional Considerations

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

In this document, the terms “computer program medium” and “computer usable medium” and “computer readable medium”, as well as variations thereof, are used to generally refer to transitory or non-transitory media. These and other various forms of computer program media or computer usable/readable media may be involved in carrying one or more sequences of one or more instructions to a processing device for execution. Such instructions embodied on the medium, may generally be referred to as “computer program code” or a “computer program product” or “instructions” (which may be grouped in the form of computer programs or other groupings). When executed, such instructions may enable a computing module, such as the analyte sensor system 208 and/or analyte sensor system 800, circuitry related thereto, and/or a processor thereof or connected thereto to perform features or functions of the present disclosure as discussed herein (for example, in connection with methods described above and/or in the claims), including for example when the same is/are incorporated into a system, apparatus, device and/or the like.

Various embodiments have been described with reference to specific example features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense. It will be appreciated that, for clarity purposes, the above description has described embodiments with reference to different functional units. However, it will be apparent that any suitable distribution of functionality between different functional units may be used without detracting from the invention. For example, functionality illustrated to be performed by separate computing devices may be performed by the same computing device. Likewise, functionality illustrated to be performed by a single computing device may be distributed amongst several computing devices. Hence, references to specific functional units are only to be seen as references to suitable means for providing the described functionality, rather than indicative of a strict logical or physical structure or organization.

Although described above in terms of various example embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the present application, whether or not such embodiments are described and whether or not such features are presented as being a part of a described embodiment. Thus, the breadth and scope of the present application should not be limited by any of the above-described example embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide illustrative instances of the item in discussion, not an exhaustive or limiting list thereof, the terms “a” or “an” should be read as meaning “at least one,” “one or more” or the like; the term “set” should be read to include one or more objects of the type included in the set; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Similarly, the plural may in some cases be recognized as applicable to the singular and vice versa. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic, circuitry, or other components, may be combined in a single package or separately maintained and may further be distributed in multiple groupings or packages or across multiple locations.

Additionally, the various embodiments set forth herein are described in terms of example block diagrams, flow charts, and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration. Moreover, the operations and sub-operations of various methods described herein are not necessarily limited to the order described or shown in the figures, and one of skill in the art will appreciate, upon studying the present disclosure, variations of the order of the operations described herein that are within the spirit and scope of the disclosure. It will be understood that each block of the flowchart illustrations, and combinations of blocks in the flowchart illustrations, may be implemented by execution of computer program instructions. These computer program instructions may be loaded onto a computer or other programmable data processing apparatus (such as a controller, microcontroller, microprocessor or the like) in a sensor electronics system to produce a machine, such that the instructions which execute on the computer or other programmable data processing apparatus create instructions for implementing the functions specified in the flowchart block or blocks. These computer program instructions may also be stored in a computer-readable memory that may direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks presented herein.

It should be appreciated that all methods and processes disclosed herein may be used in any glucose or other analyte monitoring system, continuous or intermittent. It should further be appreciated that the implementation and/or execution of all methods and processes may be performed by any suitable devices or systems, whether local or remote. Further, any combination of devices or systems may be used to implement the present methods and processes.

In addition, the operations and sub-operations of methods described herein may be carried out or implemented, in some cases, by one or more of the components, elements, devices, modules, circuitry, processors, etc. of systems, apparatuses, devices, environments, and/or computing modules described herein and referenced in various of figures of the present disclosure, as well as one or more sub—components, elements, devices, modules, processors, circuitry, and the like depicted therein and/or described with respect thereto. In such instances, the description of the methods or aspects thereof may refer to a corresponding component, element, etc., but regardless of whether an explicit reference is made, one of skill in the art will recognize upon studying the present disclosure when the corresponding component, element, etc. may be used. Further, it will be appreciated that such references do not necessarily limit the described methods to the particular component, element, etc. referred to. Thus, it will be appreciated by one of skill in the art that aspects and features described above in connection with (sub-) components, elements, devices, modules, and circuitry, etc., including variations thereof, may be applied to the various operations described in connection with methods described herein, and vice versa, without departing from the scope of the present disclosure.

What is claimed is:

1. A method, comprising:
receiving, at a computing device and during an overlap period, a first data stream of first analyte measurements from a first analyte sensor worn by a user;
receiving, at the computing device and during the overlap period, a second data stream of second analyte measurements from a second analyte sensor worn by the user, the first data stream and the second data stream received concurrently during the overlap period;
generating a combined analyte measurement based on the first analyte measurements and the second analyte measurements; and
outputting, on a display of the computing device, the combined analyte measurement.

2. The method of claim 1, wherein generating the combined analyte measurement comprises:
applying a first weight to the first analyte measurements received from the first analyte sensor;
applying a second weight to the second analyte measurements received from the second analyte sensor; and
generating the combined analyte measurement based on the weighted first analyte measurements and the weighted second analyte measurements.

3. The method of claim 2, wherein the first weight is relatively larger towards a beginning of the overlap period and relatively smaller towards an end of the overlap period.

4. The method of claim 2, wherein the second weight is relatively smaller towards a beginning of the overlap period and relatively larger towards an end of the overlap period.

5. The method of claim 2, wherein generating the combined analyte measurement comprises determining a sum of the weighted first analyte measurements and the weighted second analyte measurements.

6. The method of claim 2, wherein the generating the combined analyte measurement comprises generating a weighted average of the first analyte measurements and the second analyte measurements.

7. The method of claim 1, wherein the first analyte measurements contribute more towards the combined analyte measurement towards a beginning of an overlap period than at an end of the overlap period.

8. The method of claim 1, wherein the second analyte measurements contribute more towards the combined analyte measurement towards an end of an overlap period than at an end of the overlap period.

9. The method of claim 1, wherein the generating the combined analyte measurement further comprises:
determining which of the first analyte measurements and the second analyte measurements are more accurate; and
outputting the more accurate analyte measurements.

10. The method of claim 9, wherein less accurate analyte measurements are not output.

11. The method of claim 9, wherein less accurate analyte measurements are output concurrently with the more accurate analyte measurements, and wherein the less accurate analyte measurements include an indication that they are less accurate.

12. The method of claim 1, wherein the first analyte sensor and the second analyte sensor comprise glucose sensors.

13. The method of claim 1, wherein at least one of the first analyte sensor and the second analyte sensor comprise a glucose sensor.

14. A method, comprising:
receiving, at a computing device, a first data stream of first analyte measurements from a first analyte sensor worn by a user;
monitoring the first analyte sensor to determine a time to apply a second analyte sensor;
based on the determined time, outputting a first notification prompting the user to apply a second analyte sensor while continuing to wear the first analyte sensor;
receiving, at the computing device and during an overlap period, the first data stream of first analyte measurements from the first analyte sensor worn by the user and a second data stream of second analyte measurements from the second analyte sensor worn by the user;
during the overlap period, monitoring the first analyte sensor and the second analyte sensor to determine a removal time to remove the first analyte sensor; and
based on the determined removal time, outputting a second notification prompting the user to remove the first analyte sensor.

15. The method of claim 14, wherein the first notification indicates the time to apply the second analyte sensor, and wherein the second notification indicates the time to remove the first analyte sensor.

16. The method of claim 14, wherein the time to apply the second analyte sensor is based at least in part on an amount of time for which the first analyte sensor is designed or certified to be worn.

17. The method of claim 14, wherein the time to apply the second analyte sensor is based at least in part on a sensor quality measure of the first analyte sensor.

18. The method of claim 14, wherein removal time to remove the first analyte sensor is based at least in part on a first sensor quality measure of the first analyte sensor and a second sensor quality measure of the second analyte sensor.

19. A system, comprising:
a first analyte monitoring device worn by a user, the first analyte monitoring device configured to produce first analyte measurements;
a second analyte monitoring device worn by the user, the second analyte monitoring device configured to produce second analyte measurements;
an overlap system implemented at a computing device, the overlap system configured to:
receive, during an overlap period, the first analyte measurements from the first analyte monitoring device and the second analyte measurements from the second analyte monitoring device;
generate a combined analyte measurement based on the first analyte measurements and the second analyte measurements; and
outputting, on a display of the computing device, the combined analyte measurement.

20. The system of claim 19, wherein the overlap system is further configured to:
prior to the overlap period, output a notification on the display of the computing device instructing the user to apply the second analyte monitoring device; and
during the overlap period, output a second notification on the display of the computing device instructing the user to remove the first analyte monitoring device.

21. A system, comprising:
a wireless communication interface to receive wirelessly and concurrently, during an overlap period, a first data stream of first analyte measurements from a first analyte sensor worn by a user and a second data stream of second analyte measurements from a second analyte sensor worn by the user;
one or more processors; and
one or more memories storing instructions that are executable by the one or more processors to perform operations including:
generating a combined analyte measurement based on the first analyte measurements and the second analyte measurements; and
causing output on a display device of the combined analyte measurement.

22. The system of claim 21, wherein the display device is configured to output the combined analyte measurement.
